(19) United States
(12) Patent Application Publication
(10) Pub. No.: US 2016/0129115 A1
(43) Pub. Date: May 12, 2016

(54) FORMULATIONS OF LIQUID STABLE ANTITHROMBIN

(71) Applicant: LFB USA, Inc., Framingham, MA (US)

(72) Inventors: Gerald R. Magneson, Needham, MA (US); Greg J. Allard, Milford, MA (US); Nicholas C. Masiello, Uxbridge, MA (US); Usha S. Patel, Milford, MA (US); Lauren K. Fontana, Cumberland, RI (US); Joseph L. Pearce, Plainville, MA (US); Jerry A. Grandoni, Ashland, MA (US)

(73) Assignee: LFB USA, Inc., Framingham, MA (US)

(21) Appl. No.: 14/745,276

(22) Filed: Jun. 19, 2015

Related U.S. Application Data
(63) Continuation of application No. 12/773,000, filed on May 3, 2010, now abandoned.
Application No. 12/773,000 is a continuation-in-part of application No. PCT/US2010/001064, filed on Apr. 9, 2010.
(60) Provisional application No. 61/212,379, filed on Apr. 10, 2009.

(51) Int. Cl. A61K 47/26; A61K 47/12; A61K 47/02; A61K 38/57

(52) U.S. Cl. CPC A61K 47/26; A61K 38/57; A61K 47/12; A61K 47/02

(57) ABSTRACT

In one aspect, the invention provides liquid stable formulations of antithrombin.

### RELATED APPLICATIONS

This application is a continuation-in-part of International application PCT/US2010/001064 designating the United States, filed on Apr. 9, 2010, which claims the benefit under 35 U.S.C. §119(e) of U.S. provisional application Ser. No. 61/212,379, filed on Apr. 10, 2009, the entire contents of each of which are herein incorporated by reference.

### FIELD OF THE INVENTION

The invention provides liquid formulations of antithrombin, preferably therapeutic antithrombin. Such liquid formulations are stable and can allow for prolonged storage.

### BACKGROUND OF THE INVENTION

The stability of therapeutic proteins is a general problem in the pharmaceutical industry and requires a solution specific for each therapeutic protein. Depending on the protein, individual formulation ingredients may have a great influence on the stability, and the formulation ingredients may also depend on the planned storage form and storage temperature.

Proteins are frequently lyophilized with addition of certain excipients and stored in dry form. In these cases, a loss of stability during drying has to be minimized. Also, no loss of activity should occur on reconstitution. Possible problems associated with reconstitution are, for example, flocculation or cloudiness, or the long duration before the protein has been completely dissolved, especially at high protein concentrations. Accordingly, it is advantageous to store therapeutic proteins in liquid form as the therapeutic protein does not need to be reconstituted. A problem with storage in liquid form, however, is developing a liquid formulation of a therapeutic protein that facilitates the stability of the therapeutic protein under commonly available storage conditions.

### SUMMARY OF THE INVENTION

In one aspect, stable liquid formulations of antithrombin are provided. In one embodiment, the antithrombin is therapeutic antithrombin. In still another embodiment, the antithrombin is antithrombin alfa. In another embodiment, the antithrombin is recombinant human antithrombin (rhAT). In another embodiment, the antithrombin is ATryn®. In one embodiment, the stable liquid formulation of antithrombin allows for the prolonged storage of the antithrombin without compromising the integrity or efficacy of the antithrombin. In one embodiment, the stable liquid formulation is stable for at least one year. In another embodiment, the stable liquid formulation is stable for at least two years. In still another embodiment, the stable liquid formulation is stable for more than two years. In one embodiment, the length of stability is determined by extrapolation from an Arrhenius plot.

In one embodiment, the stable liquid formulation of antithrombin is pharmaceutically acceptable. In another embodiment, the stable liquid formulation of antithromin is sterile.

In another embodiment, the stable liquid formulation of antithrombin is contained in a vial, bottle, ampoule or bag. In still another embodiment, the bag is an EVA bag. In another embodiment, the bottle is a PETG bottle.

In still another embodiment, the stable liquid formulation of antithrombin is contained in a kit. In one embodiment, the kit further comprises instructions for using the stable liquid formulation. In another embodiment, the kit further comprises a syringe. In yet another embodiment, such a kit further comprises instructions for administering the stable liquid formulation of antithrombin. In a further embodiment, the kit further comprises a solution for diluting the stable liquid formulation of antithrombin. In still another embodiment, such a kit further comprises instructions for mixing the solution for diluting the stable liquid formulation of antithrombin and the stable liquid formulation of antithrombin. The aforementioned kits are also provided in another aspect of the invention.

It was surprisingly found that a slightly basic pH has a significant stabilizing effect on minimizing latent antithrombin and soluble aggregate formation resulting in high-affinity binding to heparin. A combination of such a pH along with a sugar, such as a disaccharide, also surprisingly has a stabilizing effect. In one embodiment, the stable liquid formulation of antithrombin is one with a slightly basic pH. In another embodiment, the pH of the stable liquid formulation is between 7.5 and 8.5. In a further embodiment, the stable liquid formulation of antithrombin has a pH that is slightly basic (e.g., between 7.5 and 8.5) and a stabilizing excipient (e.g., a carboxylic acid or a salt thereof). In still a further embodiment, the stable liquid formulation of antithrombin has a pH that is slightly basic (e.g., between 7.5 and 8.5) and a salt.

In yet a further embodiment, the stable liquid formulation of antithrombin a pH that is slightly basic (e.g., between 7.5 and 8.5), a stabilizing excipient (e.g., a carboxylic acid or a salt thereof) and a salt. In yet a further embodiment, the stable liquid formulation of antithrombin has a pH that is slightly basic (e.g., between 7.5 and 8.5), a stabilizing excipient (e.g., a carboxylic acid or a salt thereof), a salt and a sugar (e.g., a disaccharide). In still a further embodiment, the stable liquid formulation of antithrombin has a pH that is slightly basic (e.g., between 7.5 and 8.5) and a sugar (e.g., a disaccharide). In still a further embodiment, the stable liquid formulation of antithrombin has a pH that is slightly basic (e.g., between 7.5 and 8.5), a sugar (e.g., a disaccharide) and stabilizing excipient (e.g., a carboxylic acid or a salt thereof). In yet a further embodiment, the stable liquid formulation of antithrombin has a pH that is slightly basic (e.g., between 7.5 and 8.5), a sugar (e.g., a disaccharide) and salt.

In one embodiment, the pH of any of the formulations is any of the pHs provided in the Figures.

It was also surprisingly found that stabilizing excipients, such as sodium citrate, has a significant stabilizing effect on minimizing latent antithrombin and soluble aggregate formation resulting in high-affinity binding to heparin. In another embodiment, the stable liquid formulation of antithrombin comprises a stabilizing excipient (e.g., a carboxylic acid or a salt thereof). In another embodiment, the stable liquid formulation of antithrombin comprises a stabilizing excipient (e.g., a carboxylic acid or a salt thereof) and has a pH that is slightly basic (e.g., between 7.5 and 8.5). In yet another embodiment, the stable liquid formulation of antithrombin comprises a stabilizing excipient (e.g., a carboxylic acid or a salt thereof) and a salt. In still another embodiment, the stable liquid formulation of antithrombin comprises a stabilizing excipient (e.g., a carboxylic acid or a salt thereof) and a sugar (e.g., a disaccharide). In a further embodiment, the stable liquid formulation of antithrombin comprises a stabilizing excipient (e.g., a carboxylic acid or a salt thereof) and a salt and has a pH that is slightly basic (e.g., between 7.5 and 8.5). In yet a further embodiment, the stable liquid formulation of antithrombin comprises a stabilizing excipient (e.g., a carboxylic acid or a salt thereof) and a sugar (e.g., a disaccharide) and has a pH that is slightly basic (e.g., between 7.5 and 8.5). In still a further embodiment, the stable liquid formulation of antithrombin comprises a stabilizing excipient (e.g., a carboxylic acid or a salt thereof), a salt and a sugar (e.g., a disaccharide). In still another embodiment, the stable liquid formulation of antithrombin comprises a stabilizing excipient (e.g., a carboxylic acid or a salt thereof), a salt and a sugar (e.g., a disaccharide) and has a pH that is slightly basic (e.g., between 7.5 and 8.5).

In one embodiment, the stabilizing excipient is citrate, succinate, tartrate, malonate, gluconate, 1,2,3,4-Butanetetracarboxylic acid (BTC), EDTA or DTPA or a salt thereof.

In one embodiment, the concentration of the stabilizing excipient of any of the formulations is any of the concentrations provided in the Figures. In another embodiment, such stabilizing excipient is any of the stabilizing excipients provided herein. In a further embodiment, the stabilizing excipient is sodium citrate. In one embodiment, the stable liquid formulation of antithrombin comprises a stabilizing excipient (e.g., a carboxylic acid or a salt thereof) and a salt and has a pH that is slightly basic (e.g., between 7.5 and 8.5). In another embodiment, such a stable liquid formulation of antithrombin further comprises a sugar (e.g., a disaccharide).

In one embodiment, the stable liquid formulation further comprises a buffer. In another embodiment, the buffer is a Tris-phosphate buffer. In a further embodiment, the buffer is at a concentration of 1 mM to 100 mM. In yet a further embodiment, the pH of the buffer is between 7.5 and 8.5. In another embodiment, the buffer is 10 mM tris-phosphate with a pH of 8.

In one embodiment, the buffer of any of the formulations provided herein is any of the buffers provided in the Figures. In another embodiments, the concentration of such buffer is any of the concentrations for the buffers provided in the Figures.

In another embodiment, the stable liquid formulation does not include a surfactant. In yet another embodiment, the stable liquid formulation further comprises a surfactant. In one embodiment, the surfactant has little (e.g., less than 5 mM, less than 4 mM, less than 3 mM, less than 2 mM or less than 1 mM hydrogen peroxide) or no hydrogen peroxide contamination. In another embodiment, the surfactant is Polysorbate 80, Polysorbate 20, Tween 20 or Tween 80. In yet another embodiment, the surfactant is 0.5 to 1% of volume by volume. In still another embodiment, the surfactant is 0.5 or 1% of volume by volume.

In one embodiment, the surfactant of any of the formulations provided herein is any of the surfactants provided in the Figures. In another embodiment, the concentration of such surfactant is any of the concentrations of the surfactants provided in the Figures.

In one embodiment, the antithrombin of the stable liquid formulation is at a concentration of 45, 50 or 55 mg/ml.

In one embodiment, the antithrombin concentration of any of the formulations provided herein is the concentration of the antithrombin provided in the Figures.

In another embodiment, the stabilizing excipient is sodium citrate. In one embodiment, the stabilizing excipient is at a concentration of 50 to 600 mM. In another embodiment, the stabilizing excipient is at a concentration of 50 to 100 mM, 50 to 150 mM, 50 to 200 mM, 50 to 250 mM, 50 to 300 mM, 50 to 350 mM, 50 to 400 mM, 50 to 450 mM, 50 to 500 mM or 50 to 550 mM. In a further embodiment, the stabilizing excipient is at a concentration of 550 to 600 mM, 500 to 600 mM, 450 to 600 mM, 400 to 600 mM, 350 to 600 mM, 300 to 600 mM, 250 to 600 mM, 200 to 650 mM, 150 to 600 mM or 100 to 600 mM. In still another embodiment, the stabilizing excipient is at a concentration of 100 to 550 mM, 150 to 500 mM, 200 to 450 mM, 250 to 400 mM or 300 to 350 mM. In another embodiment, the stabilizing excipient is at a concentration of 100, 150, 250, 500 or 600 mM.

In one embodiment, the stabilizing excipient of any of the formulations provided herein is any of the stabilizing excipients provided in the Figures. In another embodiment, the concentration of such stabilizing excipient is any of the concentrations of the stabilizing excipients provided in the Figures.

In a further embodiment, the sugar is a disaccharide sugar. In yet a further embodiment, the sugar is sucrose or trehalose. In yet another embodiment, the sugar is 0.5 to 5% of volume by weight. In still another embodiment, the sugar is 0.5, 1, 1.5, 2, 2.5, 3, 3.5, 4, 4.5 or 5% of volume by weight.

In one embodiment, the sugar of any of the formulations provided herein is any of the sugars provided in the Figures. In another embodiment, the concentration of such sugar is any of the concentrations of the sugars provided in the Figures.

In yet a further embodiment, the salt is sodium chloride. In one embodiment, the salt has a concentration of between 25 mM and 100 mM.

In one embodiment, the salt of any of the formulations provided herein is any of the salts provided in the Figures. In another embodiment, the concentration of such salt is any of the concentrations of the salts provided in the Figures.

In another embodiment, the antithrombin of the stable liquid formulation has a concentration of 50 mg/ml or 55 mg/ml, wherein the stabilizing excipient is sodium citrate and has a concentration of 300 mM, and wherein the disaccharide sugar is sucrose and is 1% of volume by weight. In a further embodiment, such a stable liquid formulation further comprises a salt (e.g., sodium chloride) and has a concentration of 50 mM. In either of these embodiments, the pH of the stable liquid formulation is 8.0. In any of these embodiments, the stable liquid formulation comprises Tris-phosphate buffer at a concentration of 10 or 20 mM.

In a further embodiment, the stable liquid formulation is any of the formulations provided in the Figures.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the Figures. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

The figures are illustrative only and are not required for enablement of the invention disclosed herein.

FIG. 1 shows the details for an example experiment.

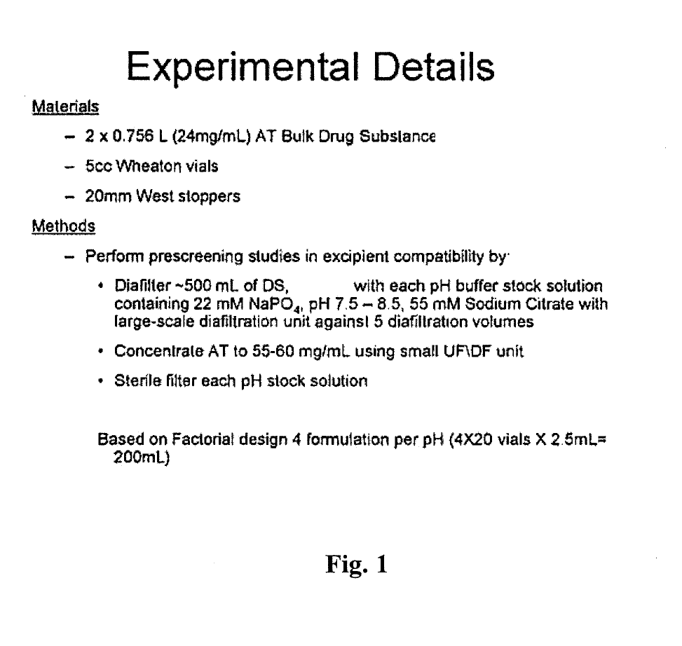

FIG. 2 shows further details of the example experiment.

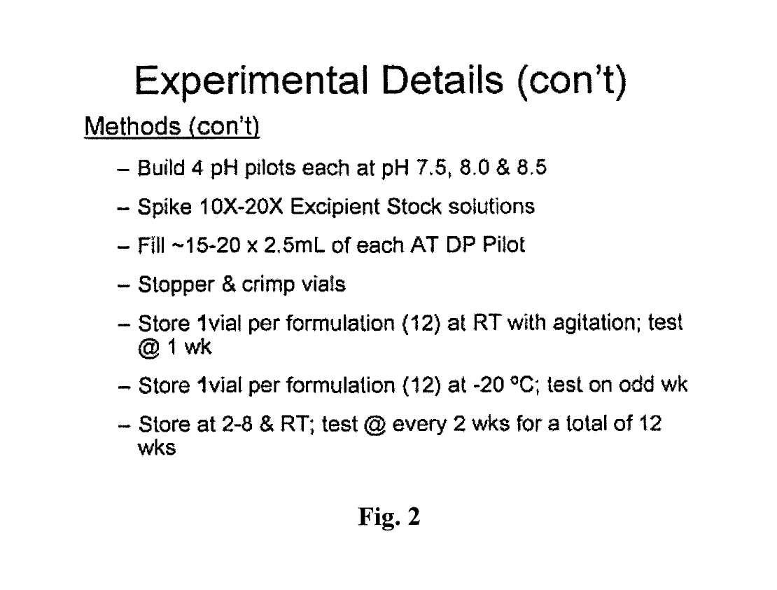

FIG. 3 shows an example plan for a formulation.

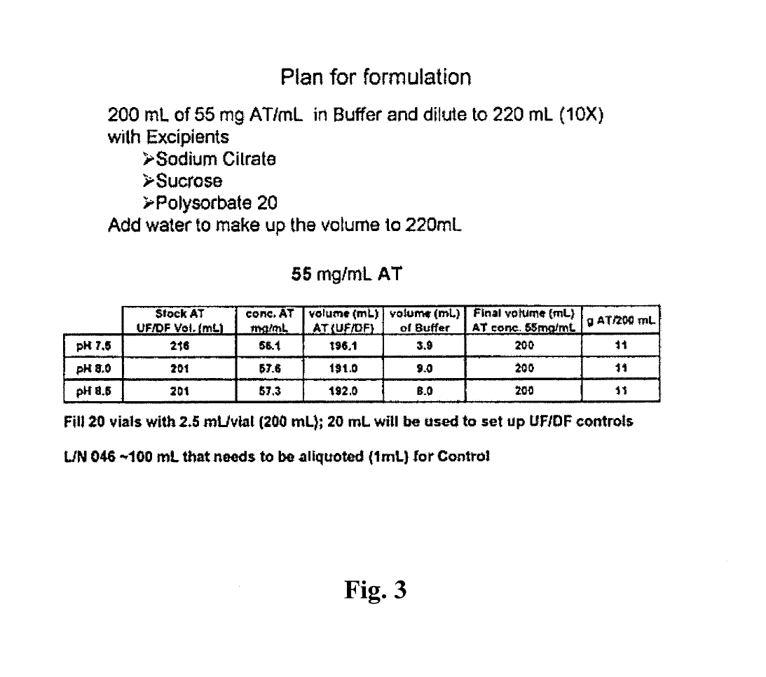

FIG. 4 provides examples of stabilizers.

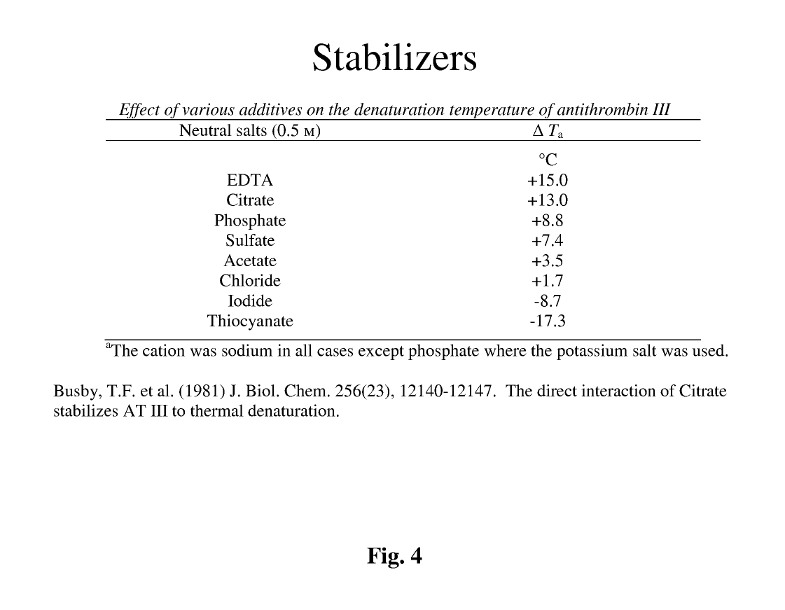

FIG. 5 provides further examples of stabilizers.

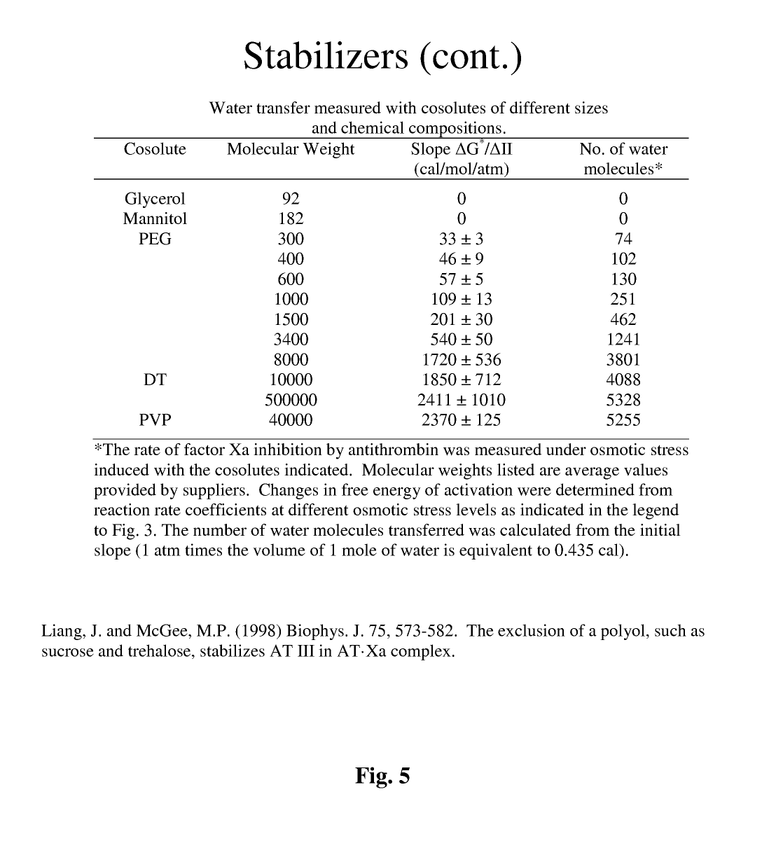

FIG. 6 provides further examples of stabilizers.

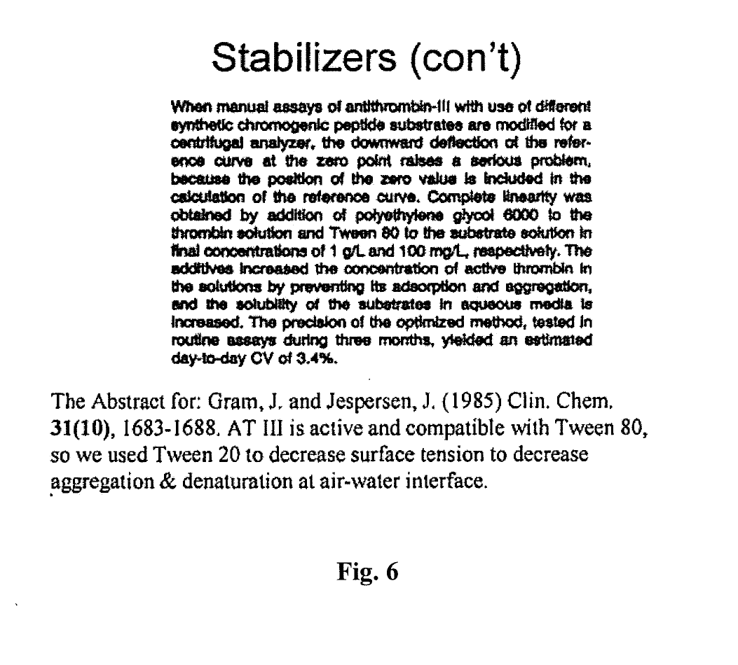

FIG. 7 provides an example of a testing plan.

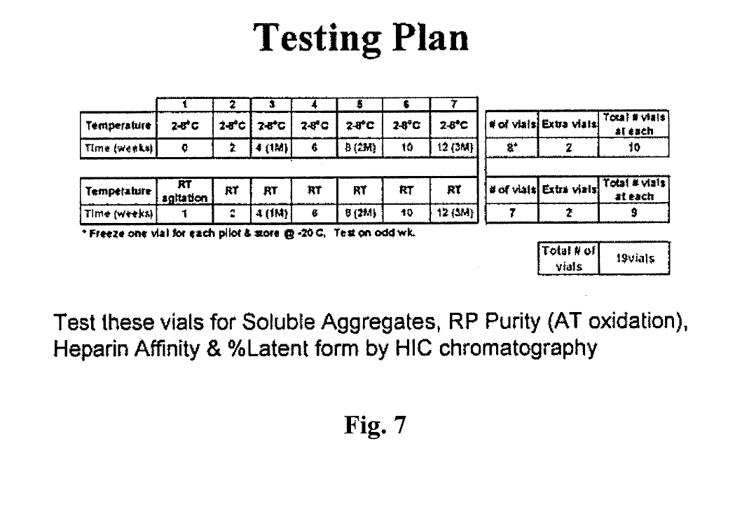

FIG. 8 provides an example of a fractional factorial design.

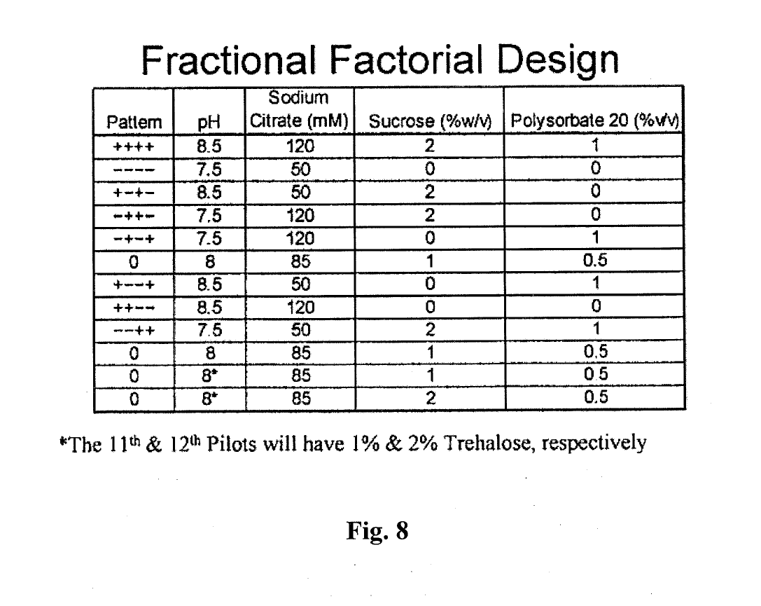

FIG. 9 provides an example of a liquid stable formulation.

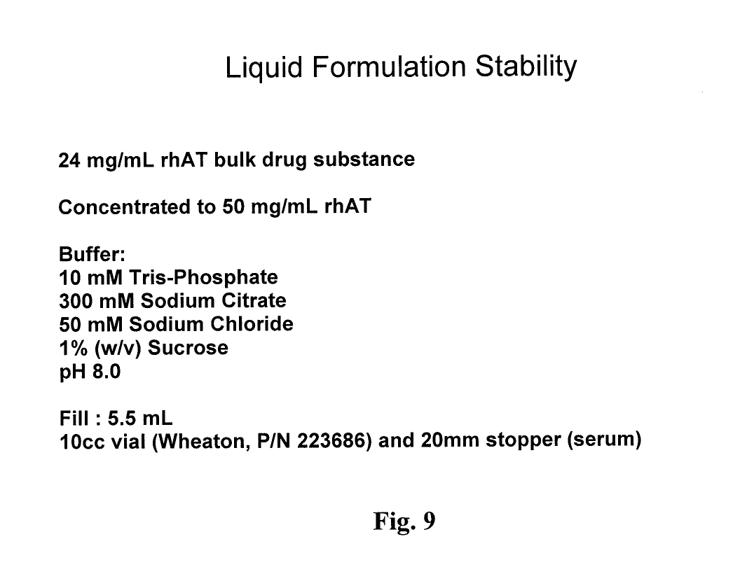

FIG. 10 provides results from analyses of the liquid stable formulation.

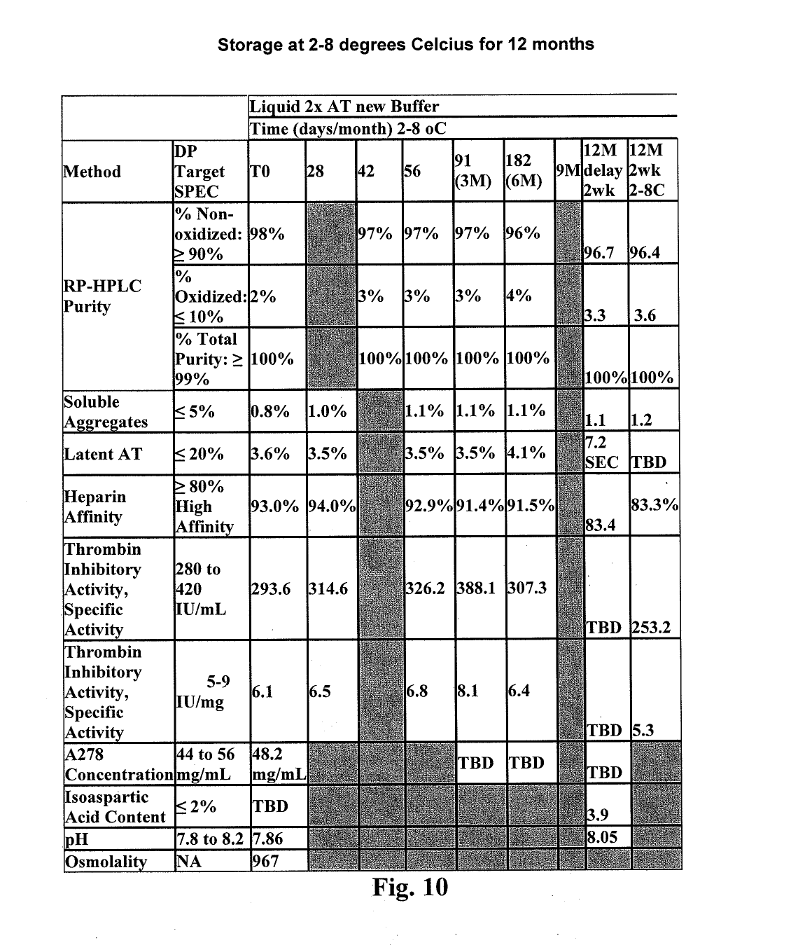

FIG. 11 provides further results from analyses of the liquid stable formulation.

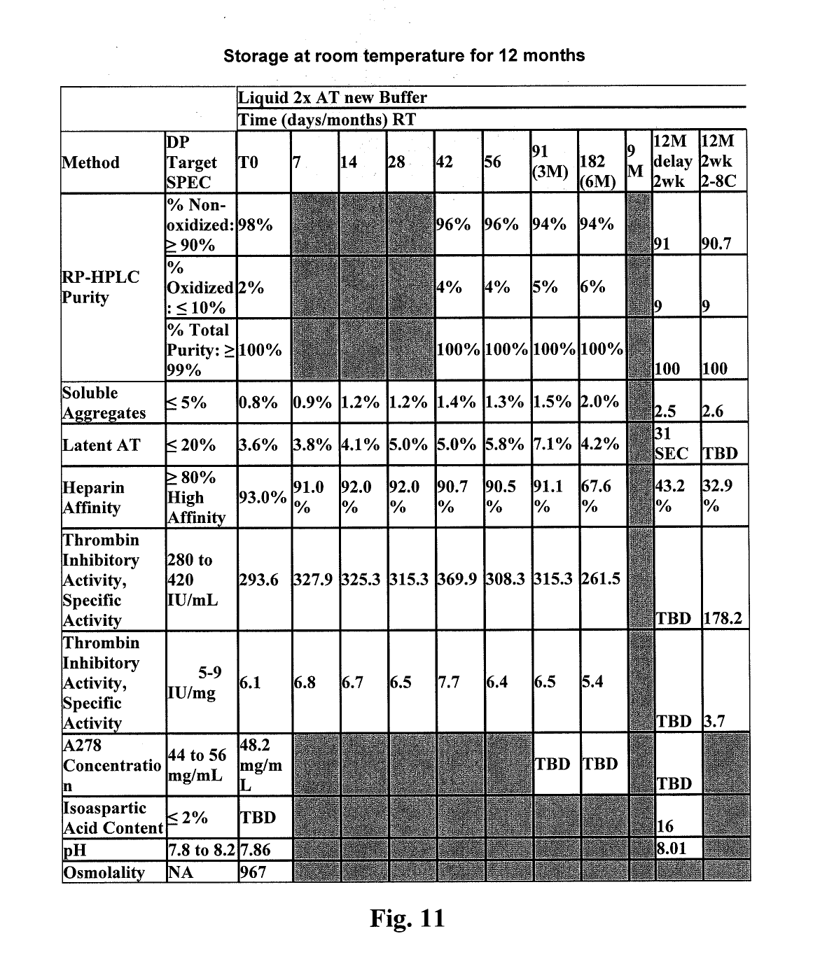

FIG. 12 provides further results from analyses of the liquid stable formulation.

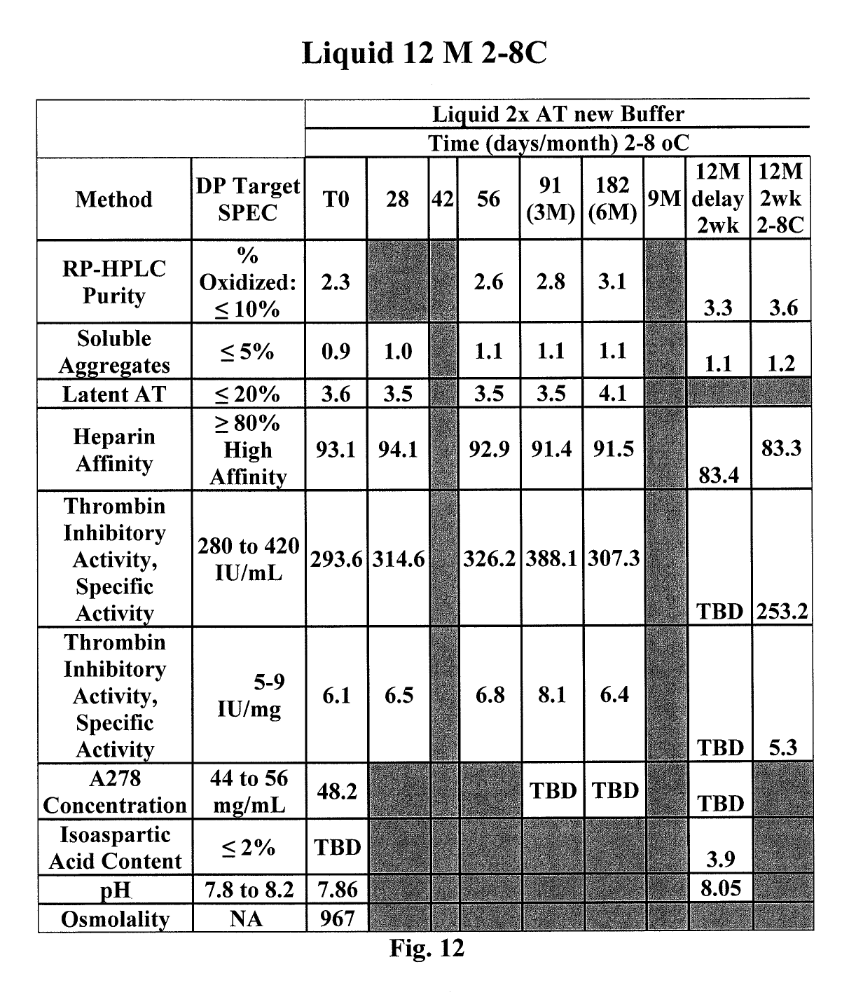

FIG. 13 provides further results from analyses of the liquid stable formulation.

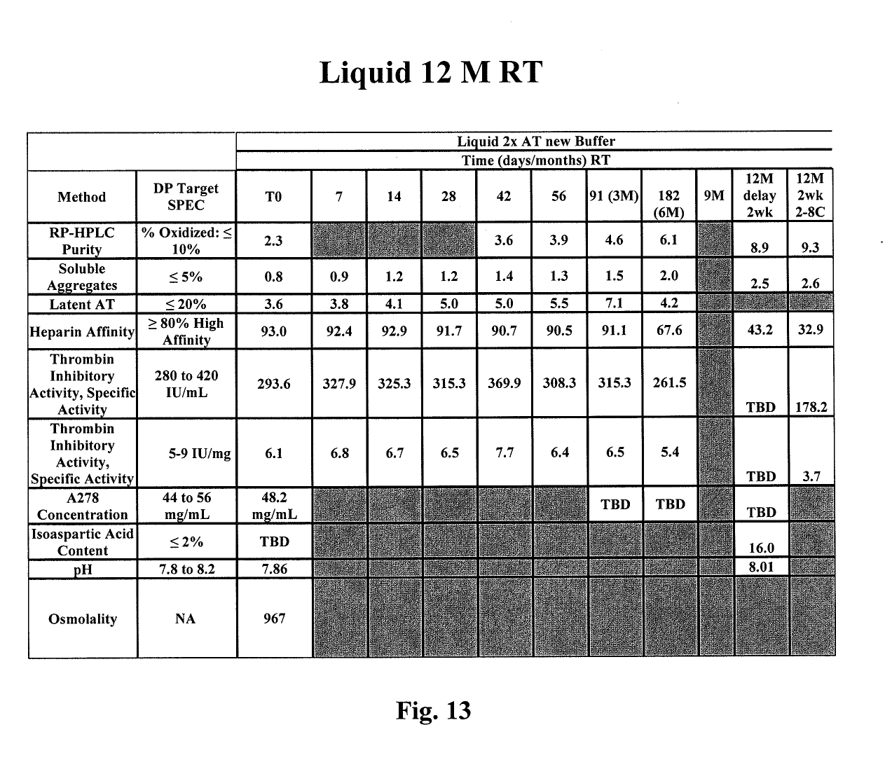

FIG. 14 provides an example assay for the measurement of latent antithrombin.

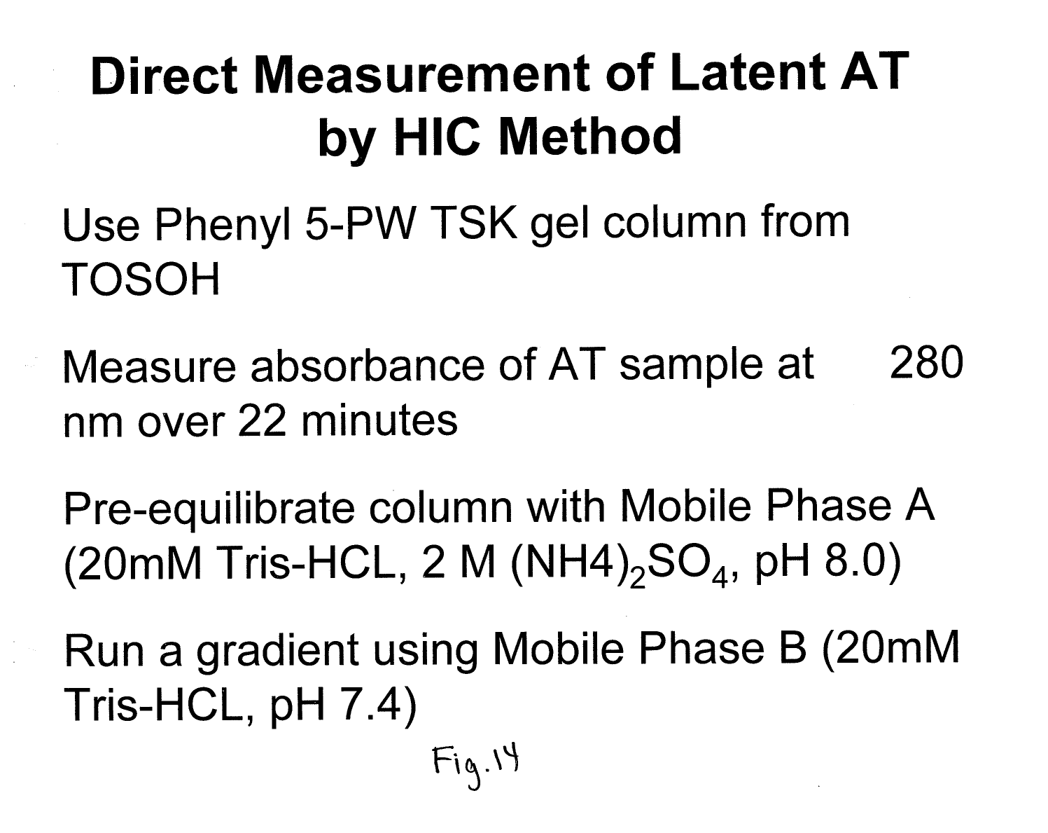

FIG. 15 provides a sample HIC chromatogram.

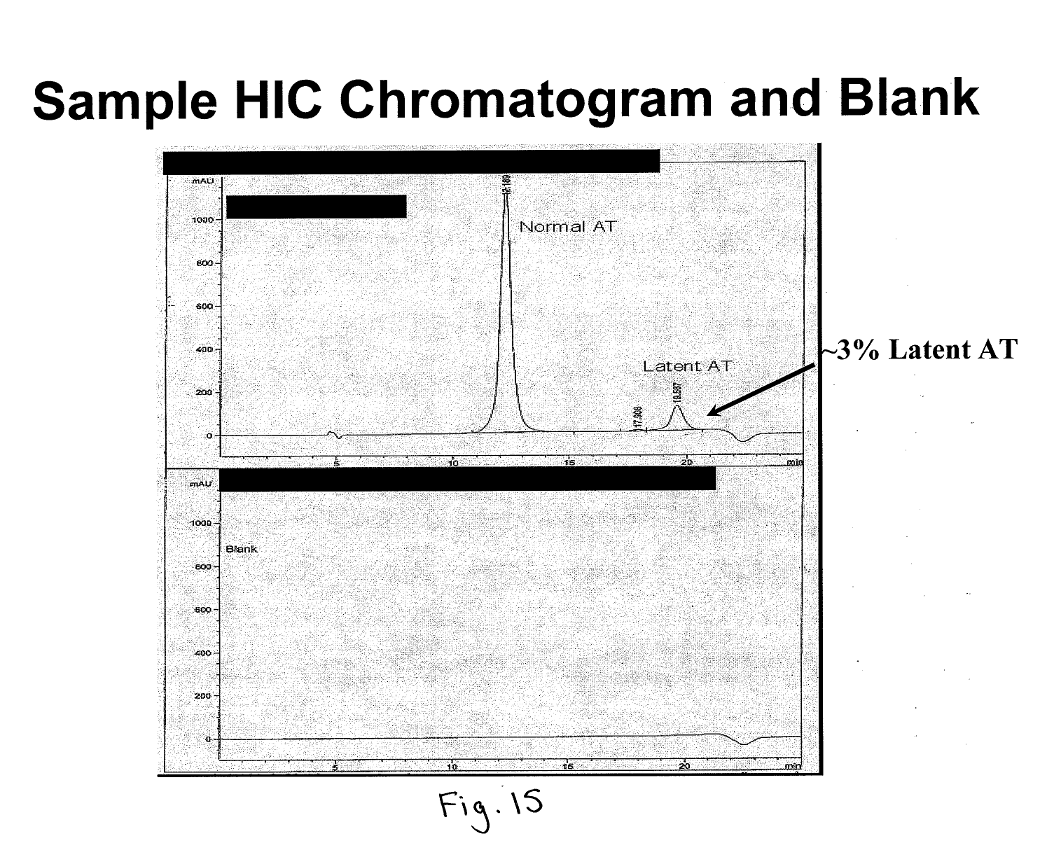

FIG. 16 provides an example of a screening study.

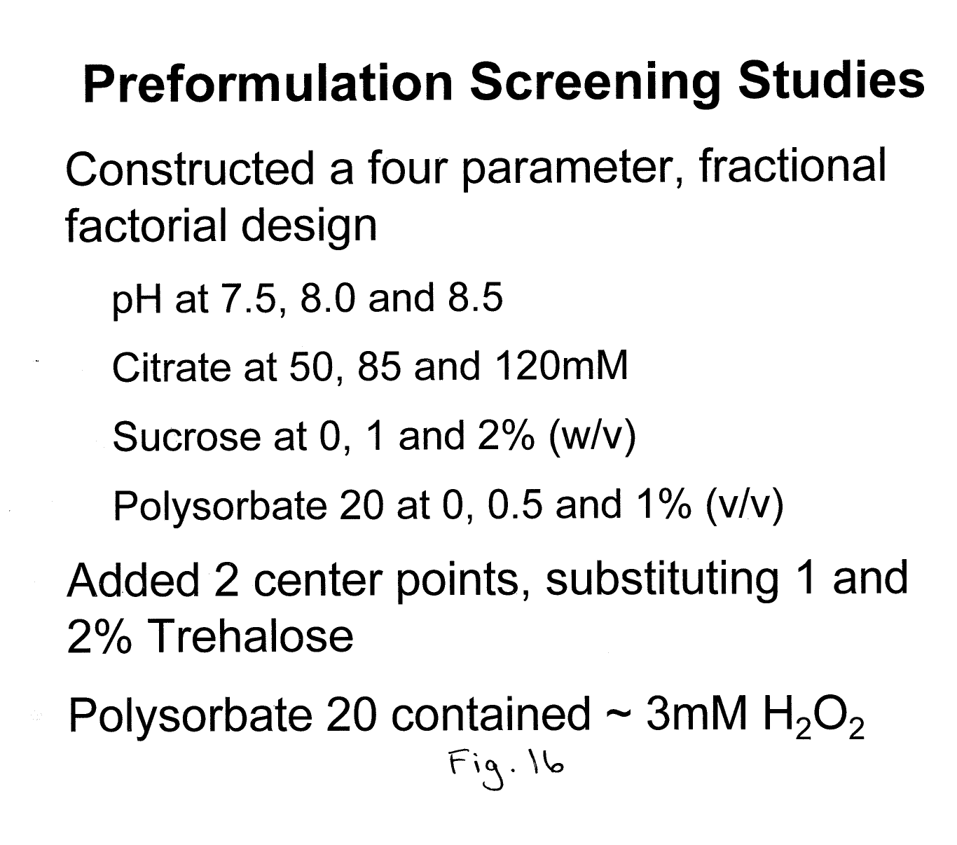

FIG. 17 provides an example of a formulation plan.

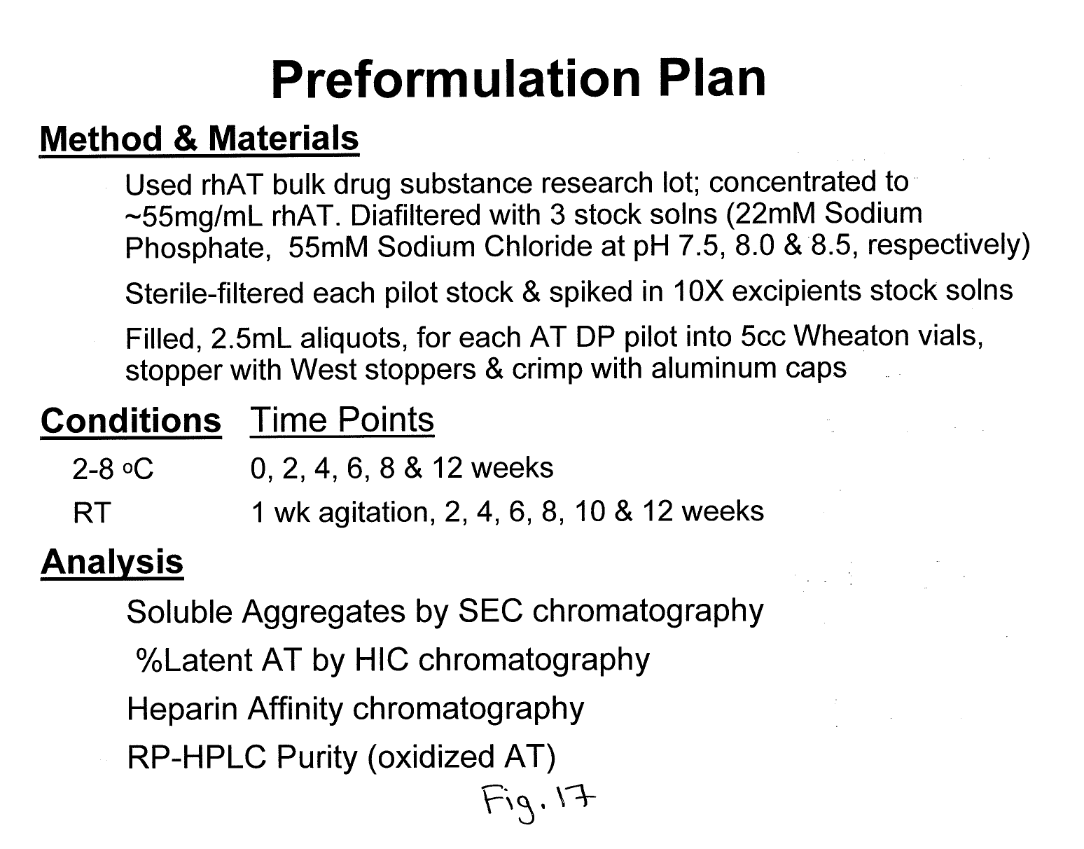

FIG. 18 provides an example of a fractional factorial design.

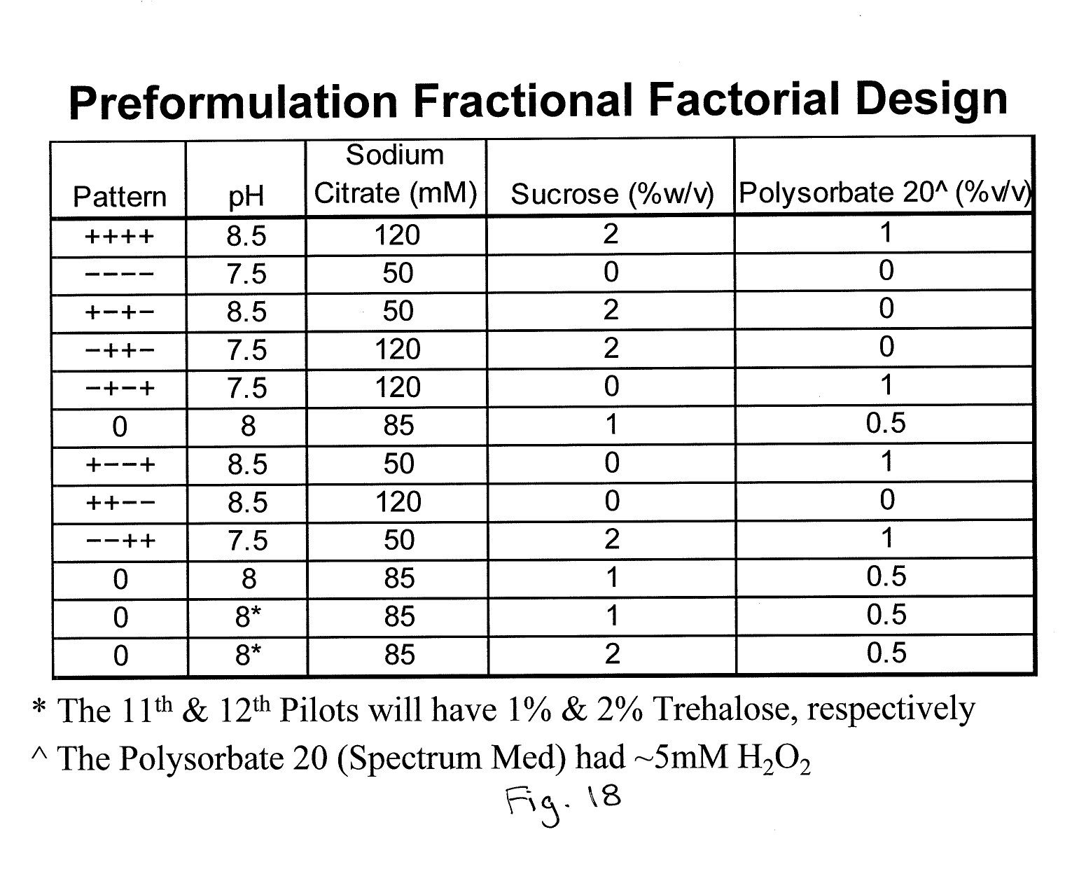

FIG. 19 provides results from an analysis of oxidized antithrombin.

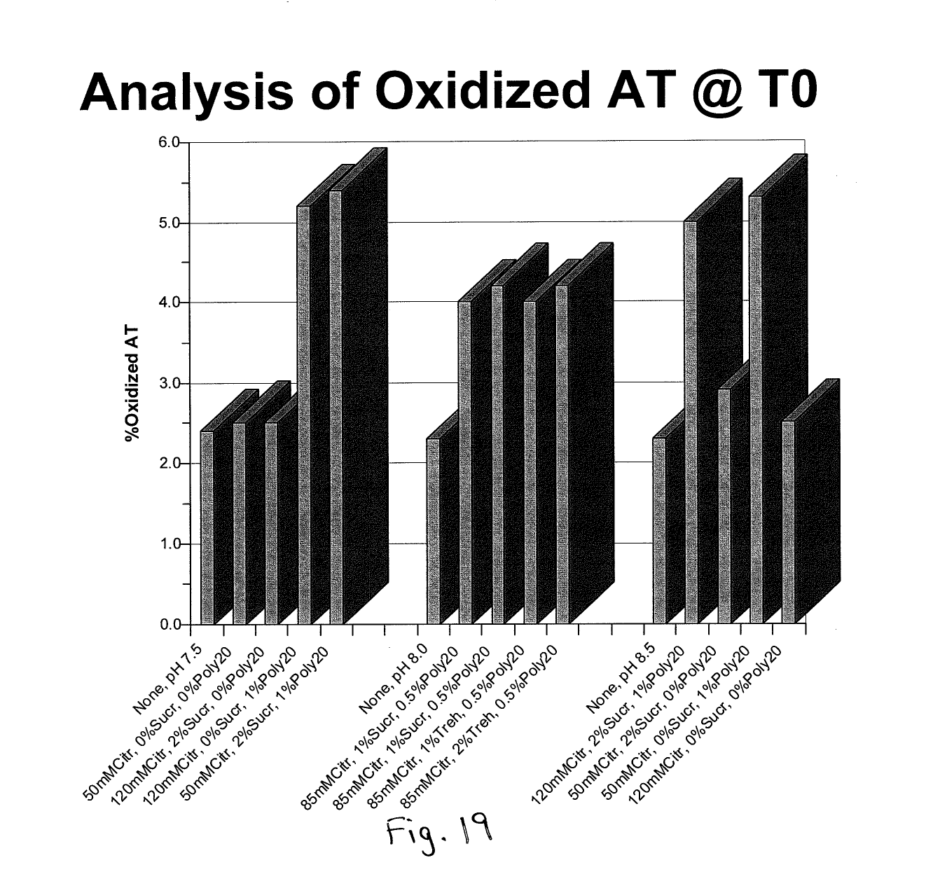

FIG. 20 provides results from an analysis of oxidized antithrombin.

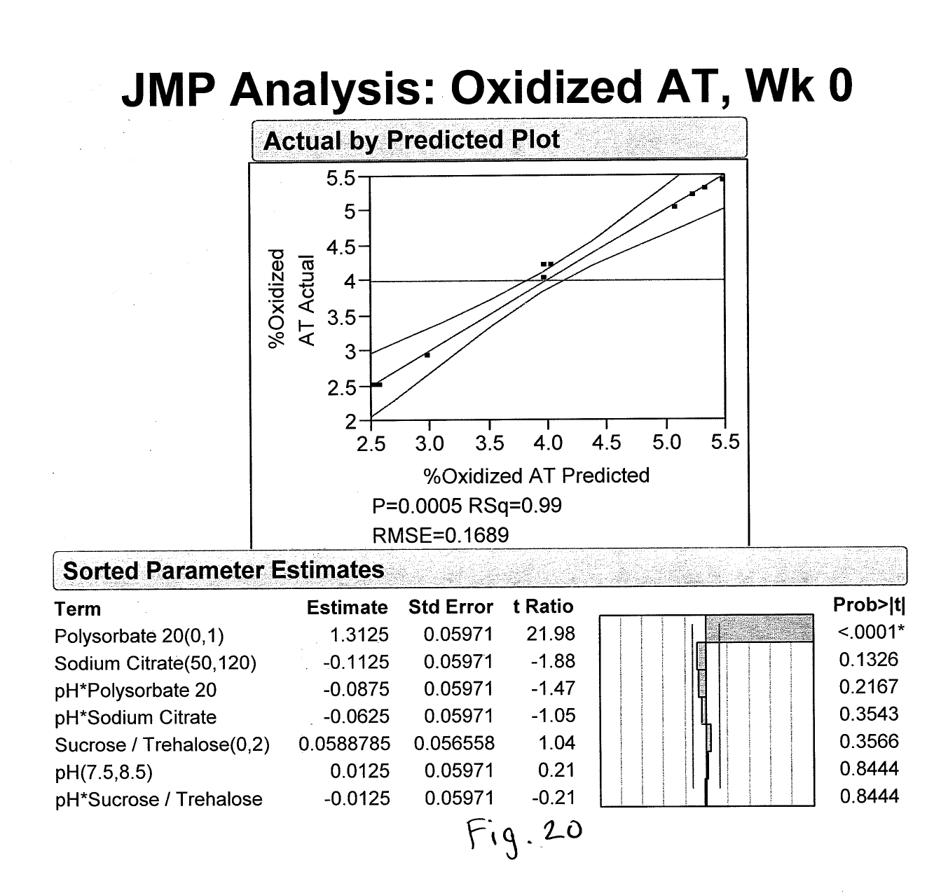

FIG. 21 provides results from an analysis of latent antithrombin.

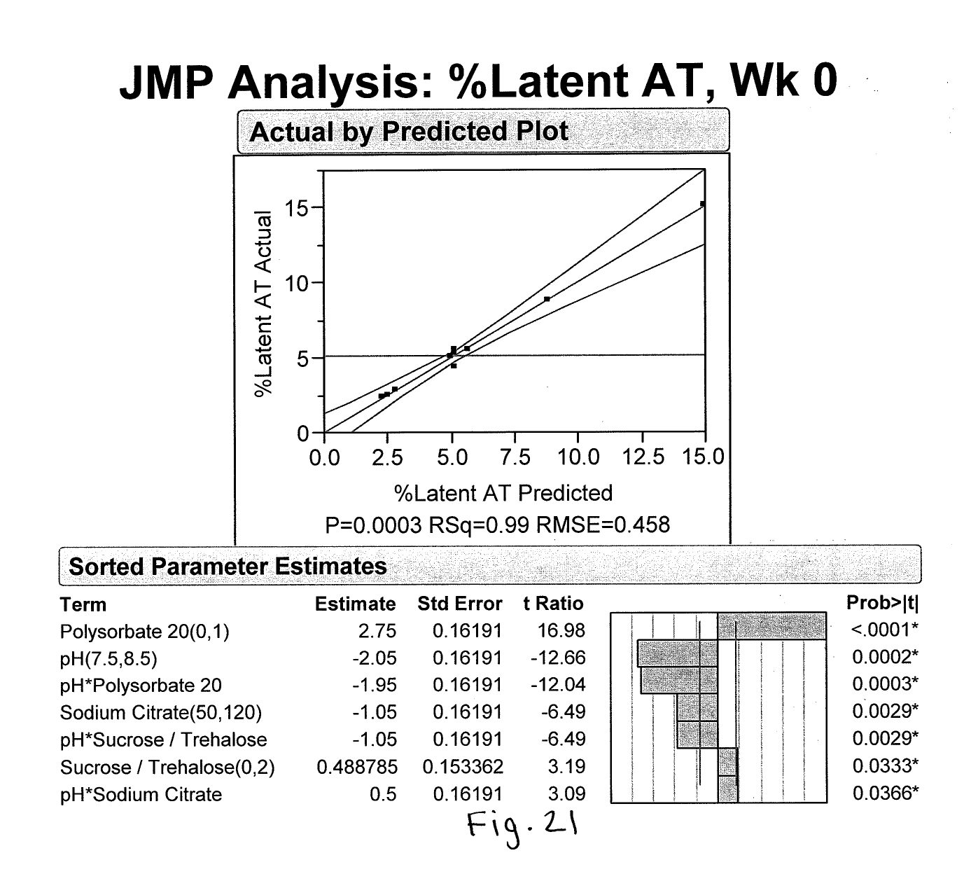

FIG. 22 provides results from an analysis of heparin affinity.

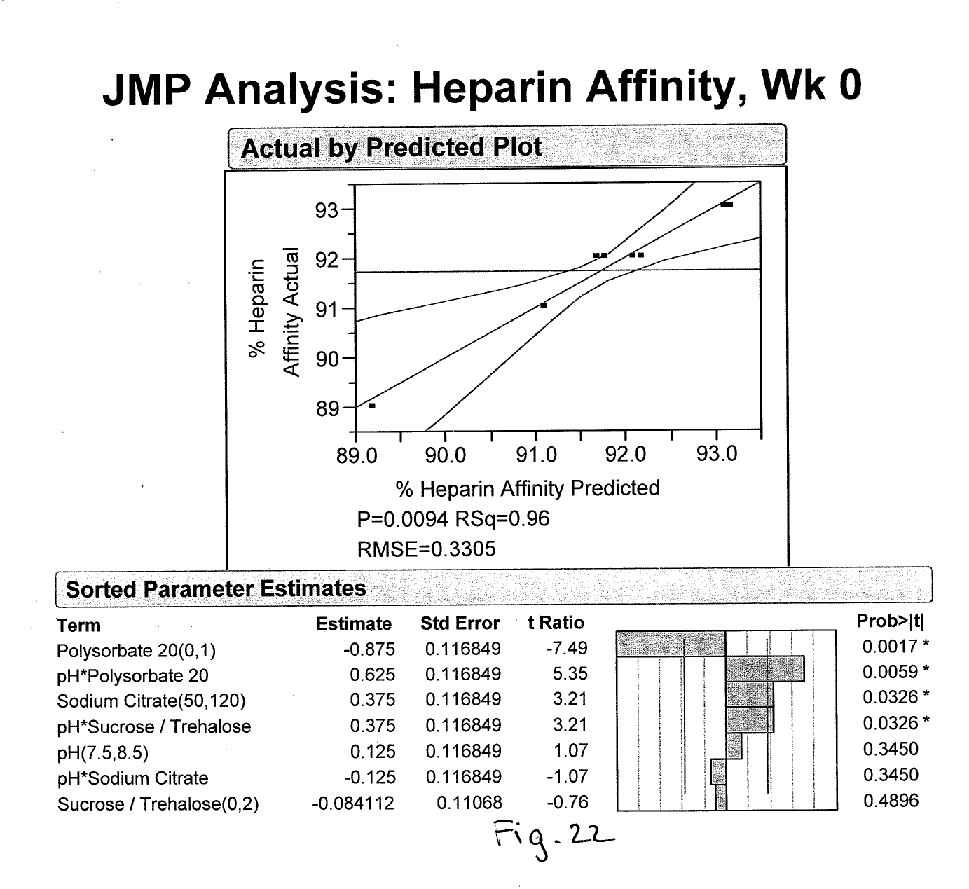

FIG. 23 provides a summary of results for three responses.

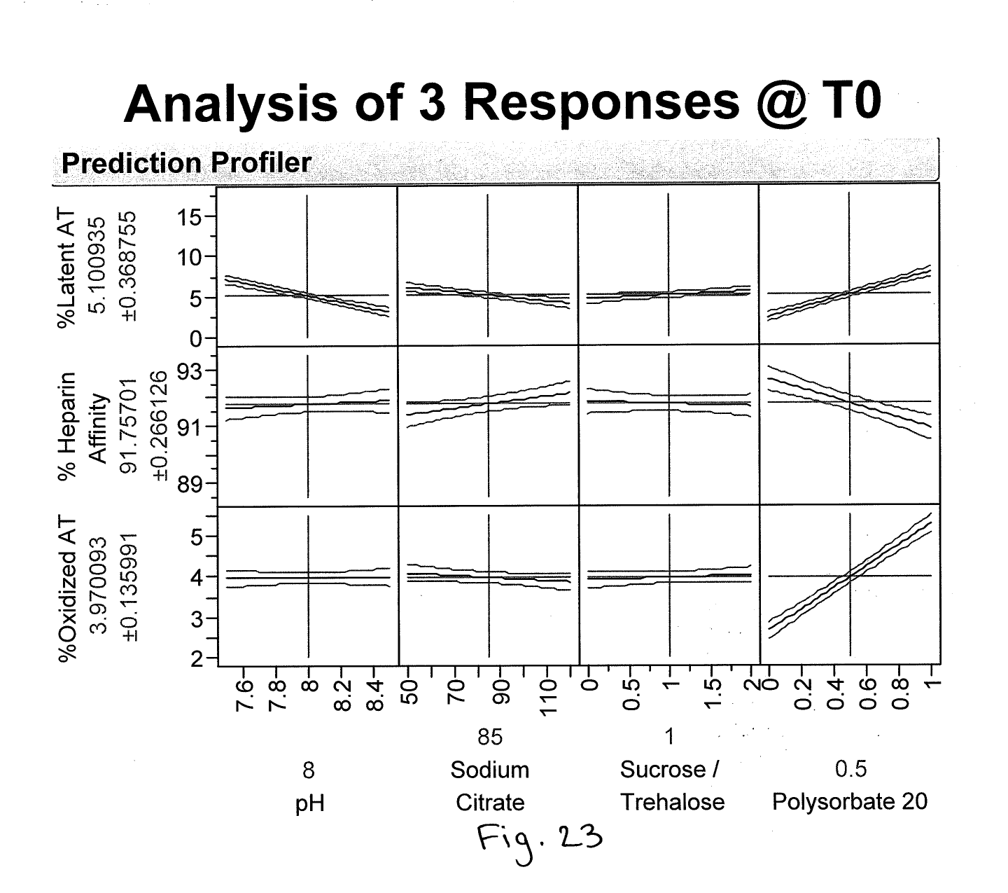

FIG. 24 provides results from an analysis of latent antithrombin.

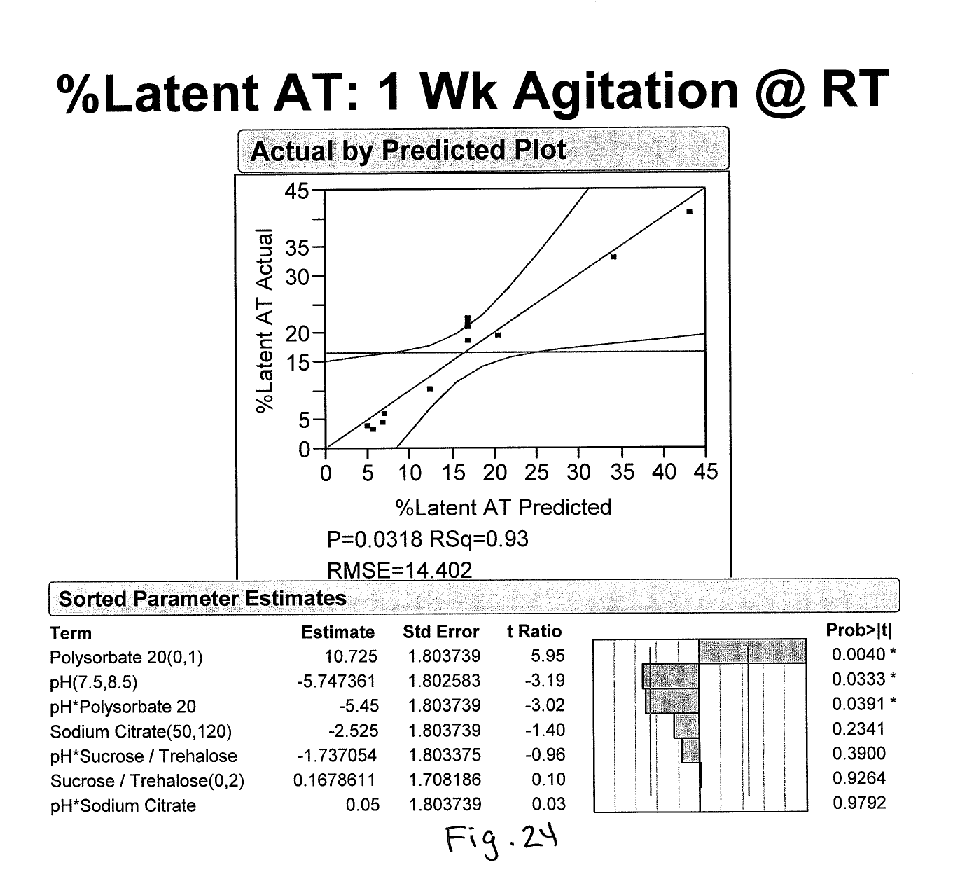

FIG. 25 provides results from an analysis of soluble aggregates.

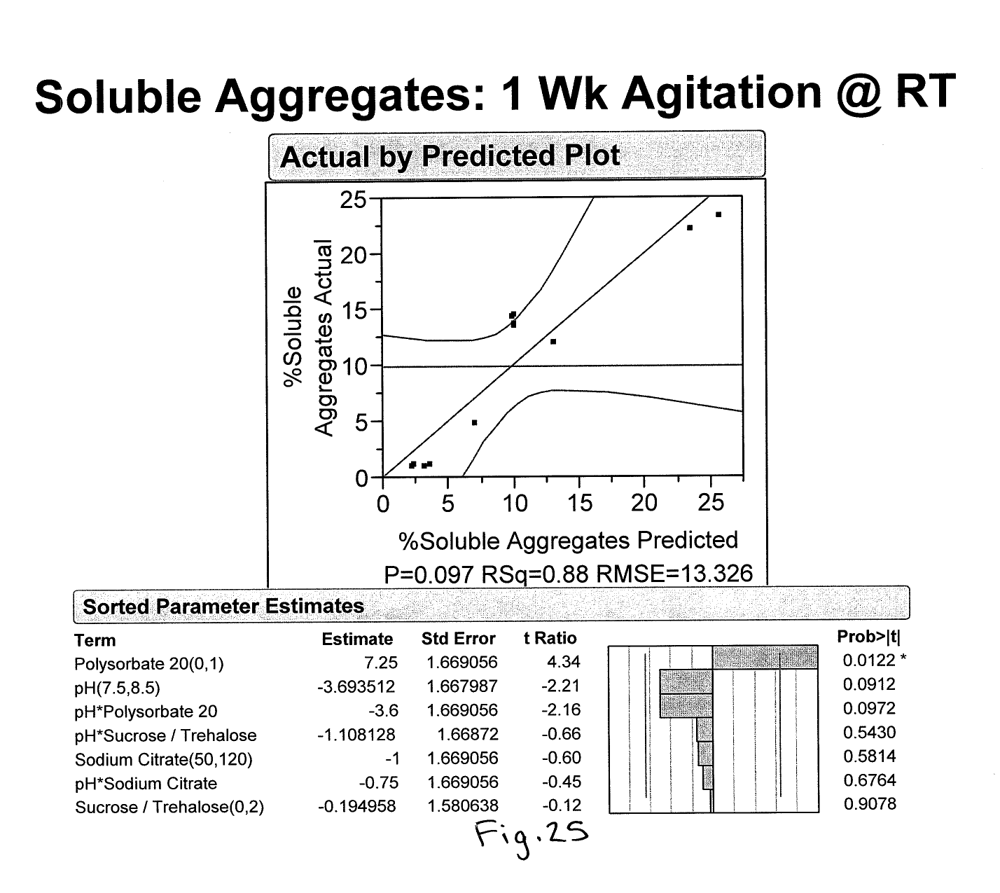

FIG. 26 provides a summary of results for four responses.

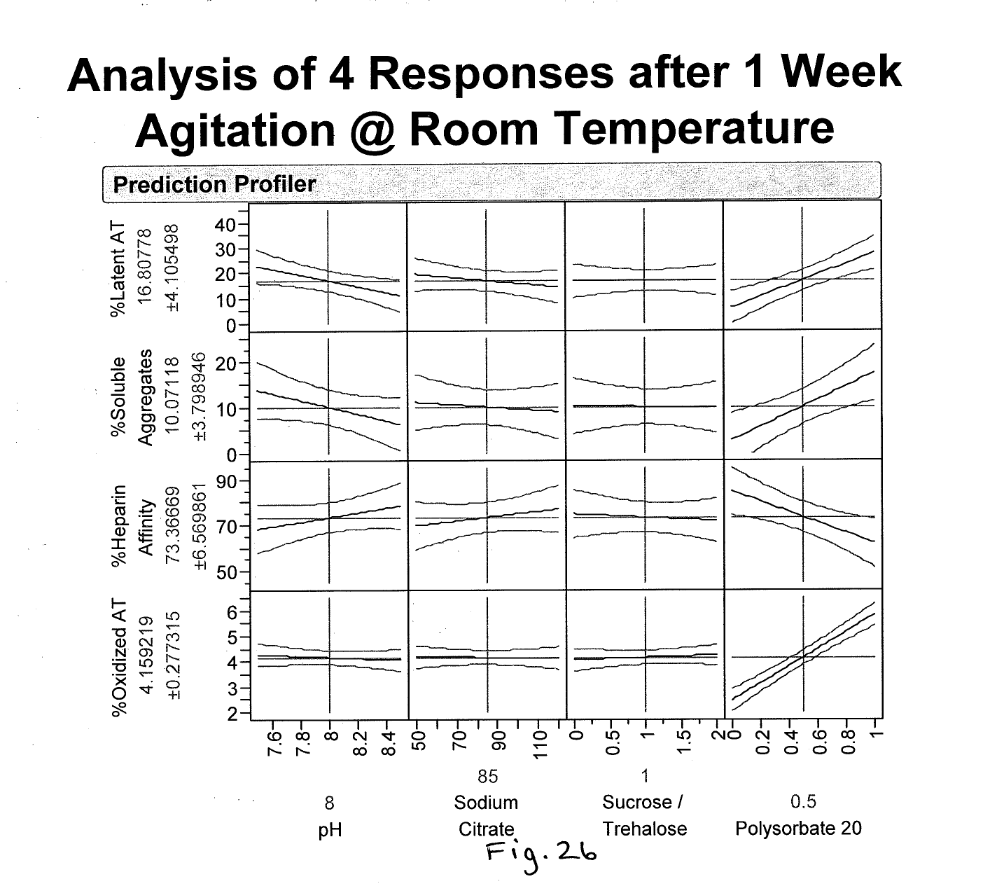

FIG. 27 provides results from an analysis of latent antithrombin.

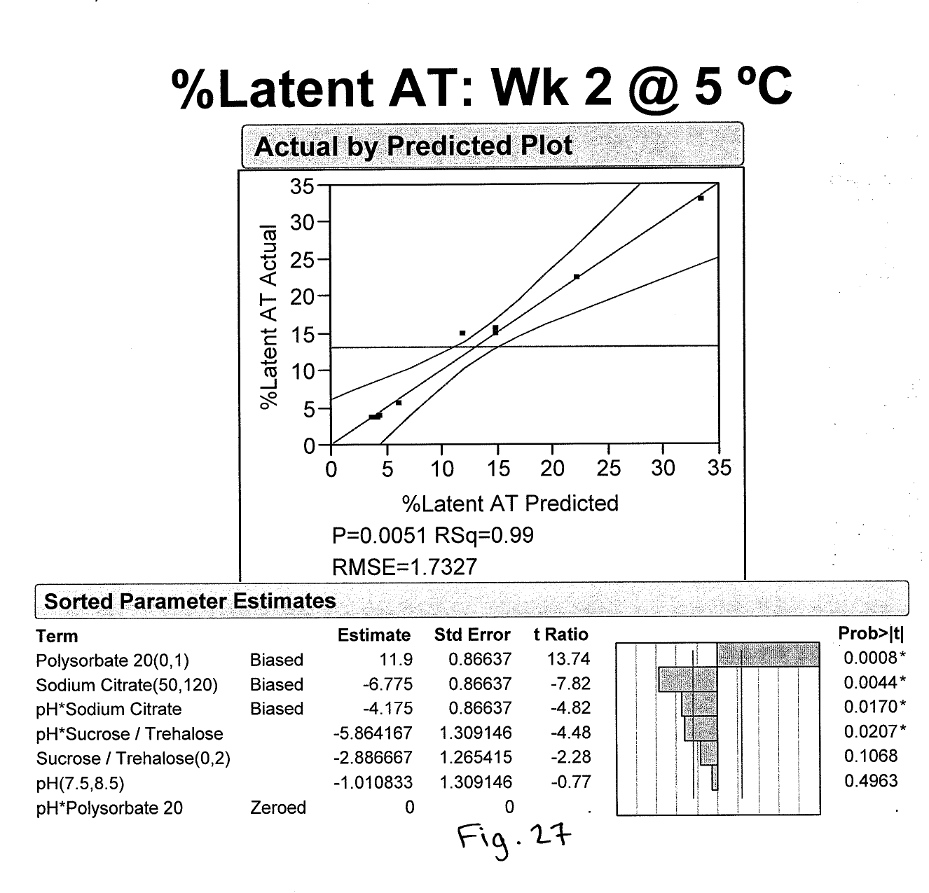

FIG. 28 describes an example of an excipient selection study.

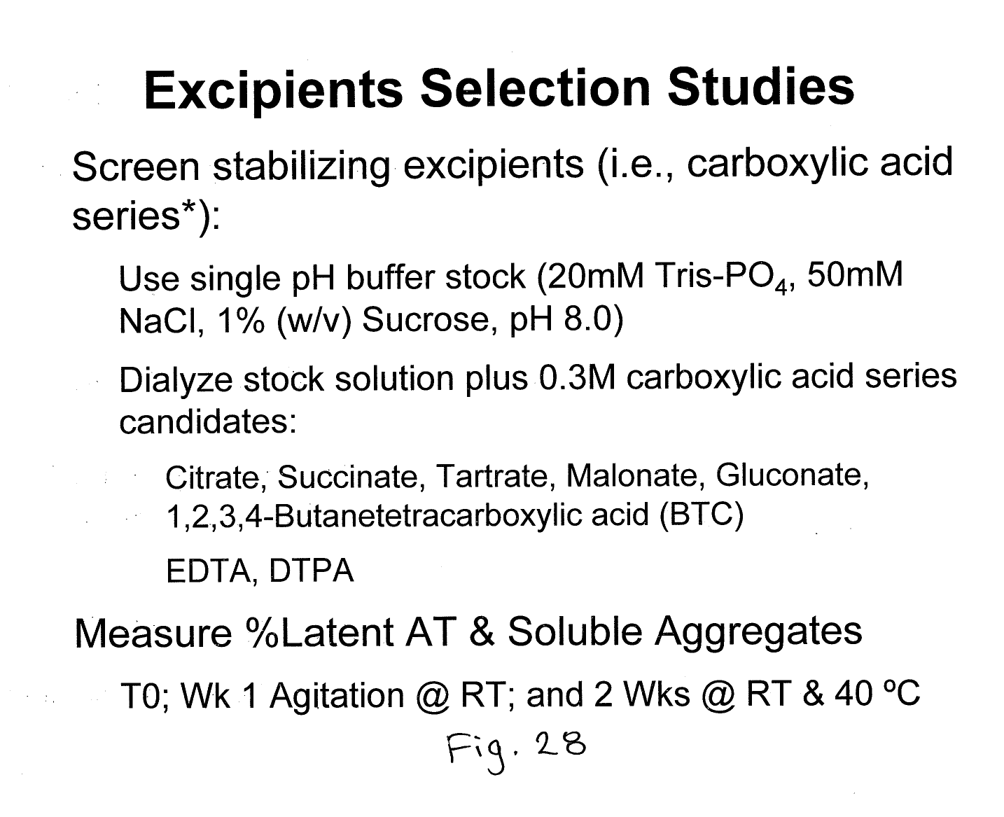

FIG. 29 provides results from an analysis of latent antithrombin.

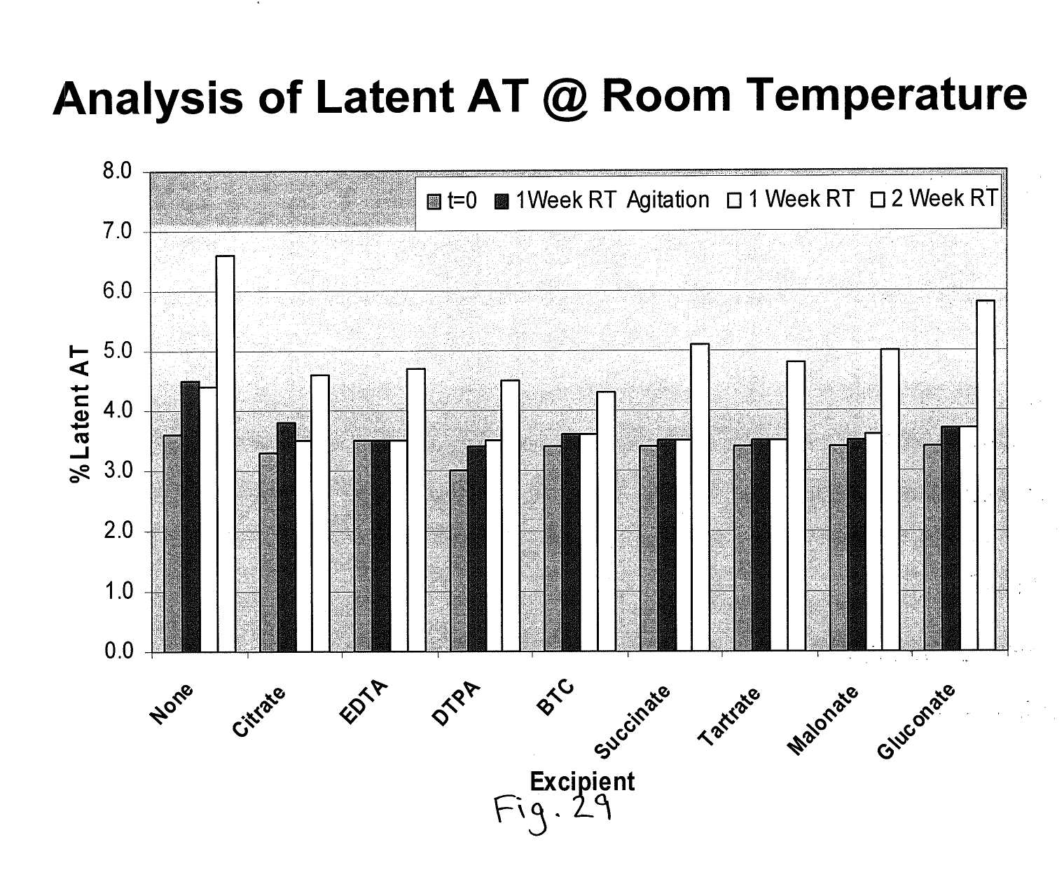

FIG. 30 provides results from an analysis of soluble aggregates.

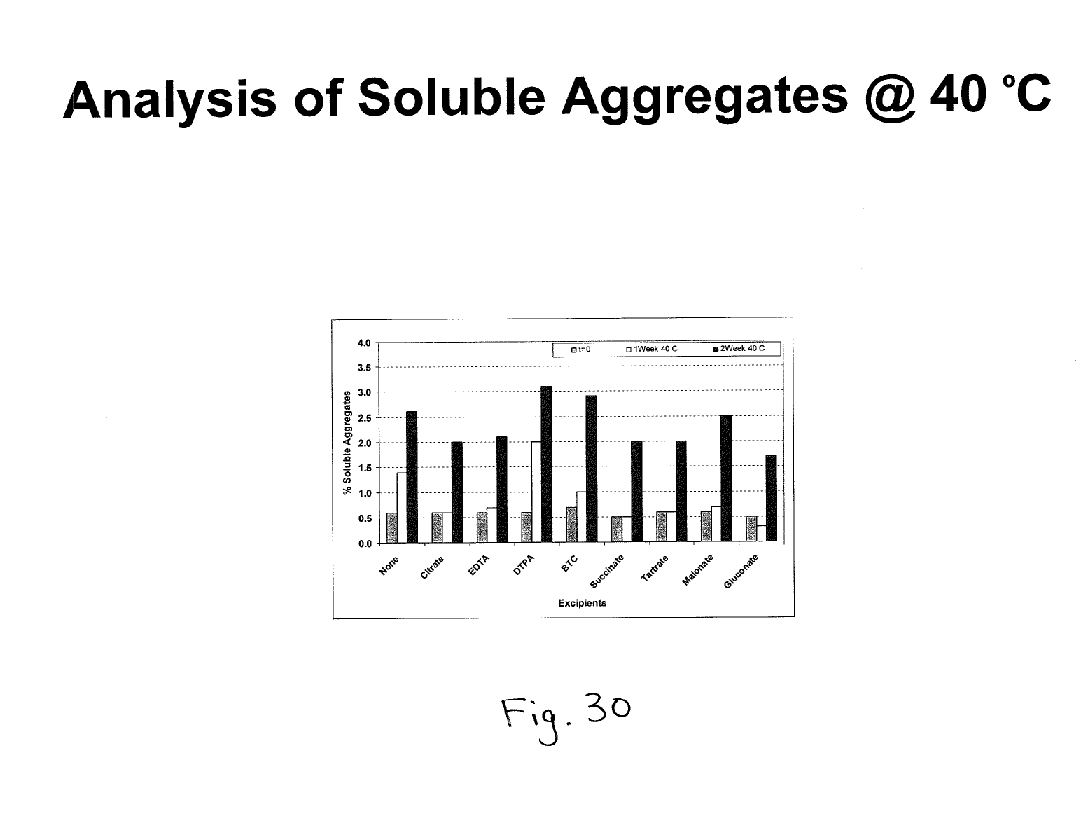

FIG. 31 provides an example of a formulation optimization study.

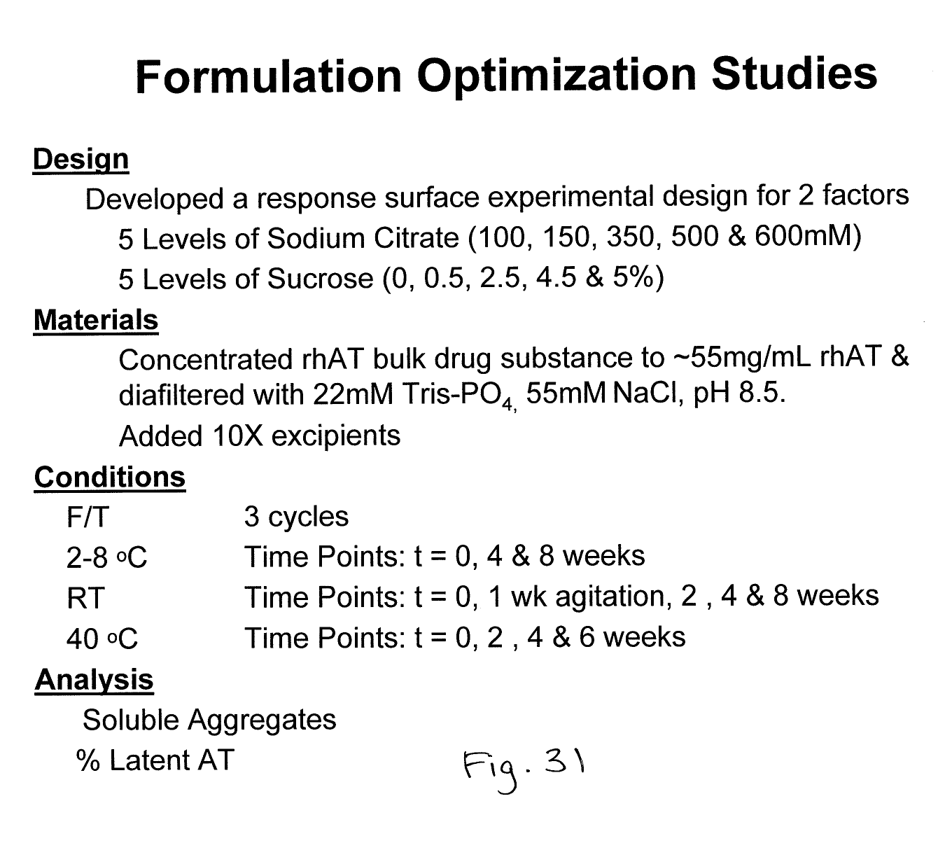

FIG. 32 provides examples of formulations for an optimization study.

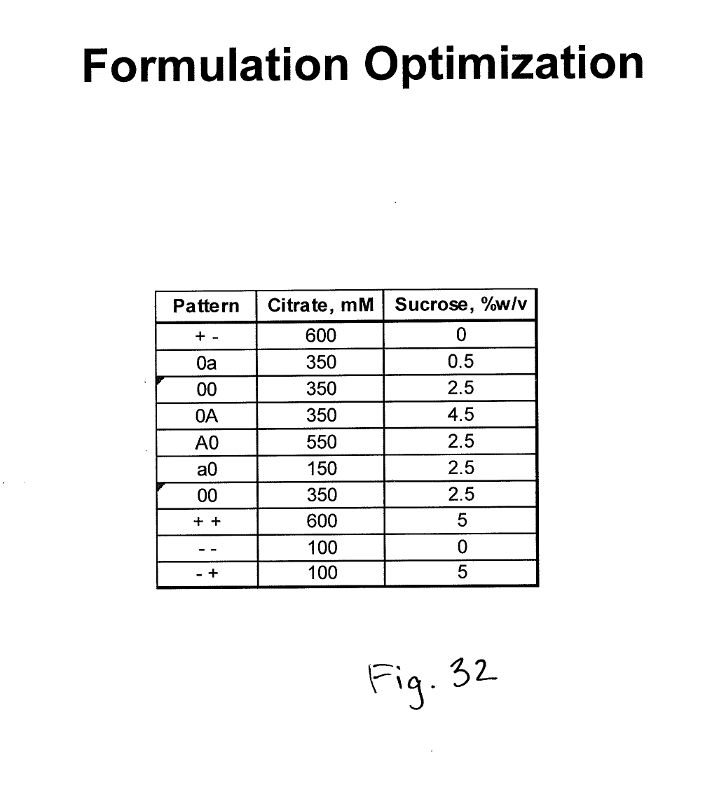

FIG. 33 shows a 3D Scatterplot of an experimental design.

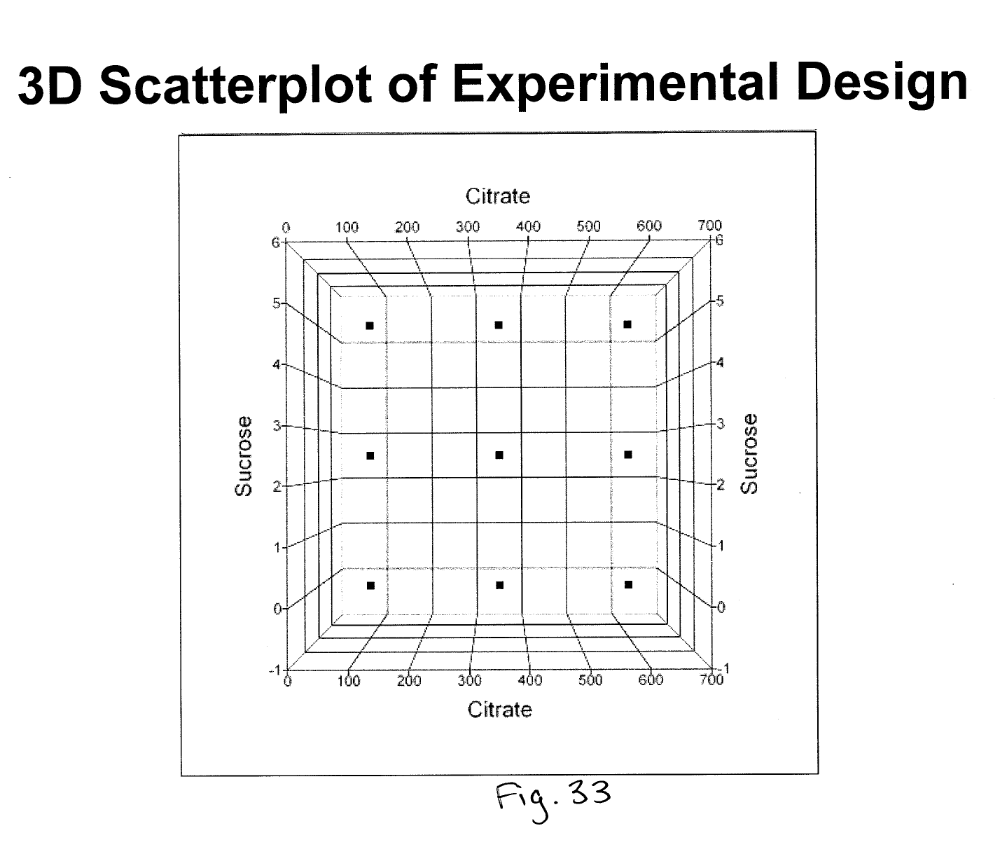

FIG. 34 provides results from an analysis of latent antithrombin.

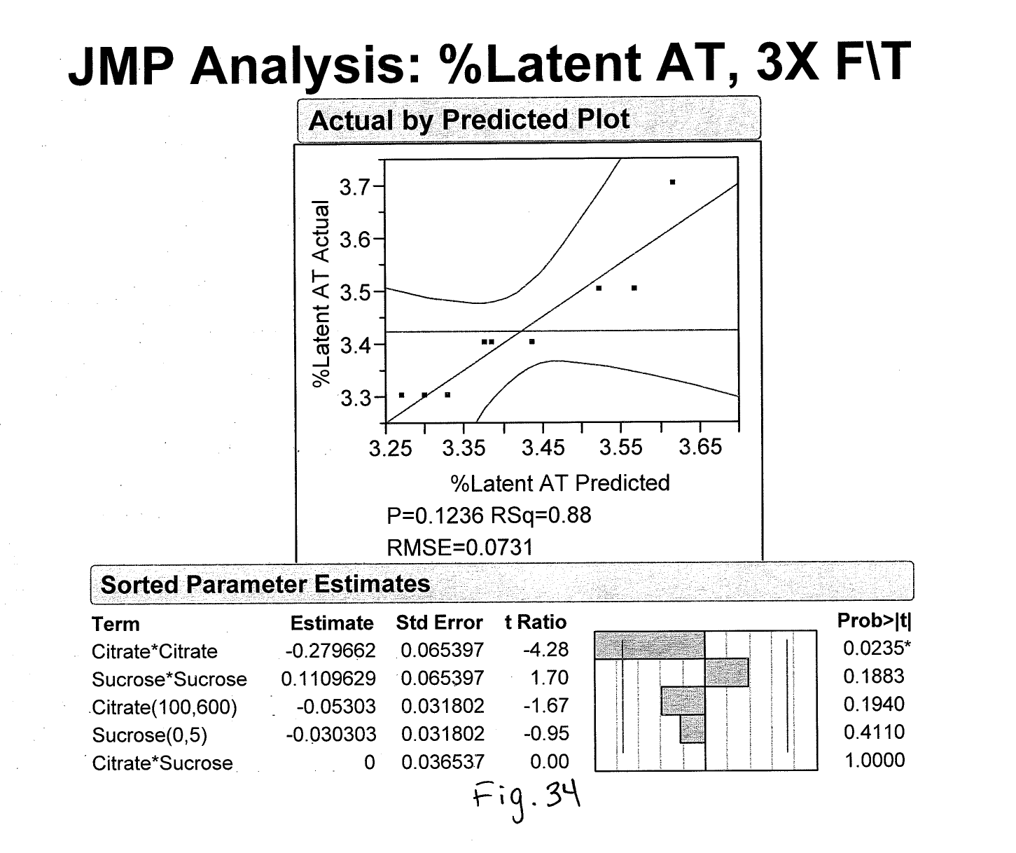

FIG. 35 provides results from an analysis of soluble aggregates.

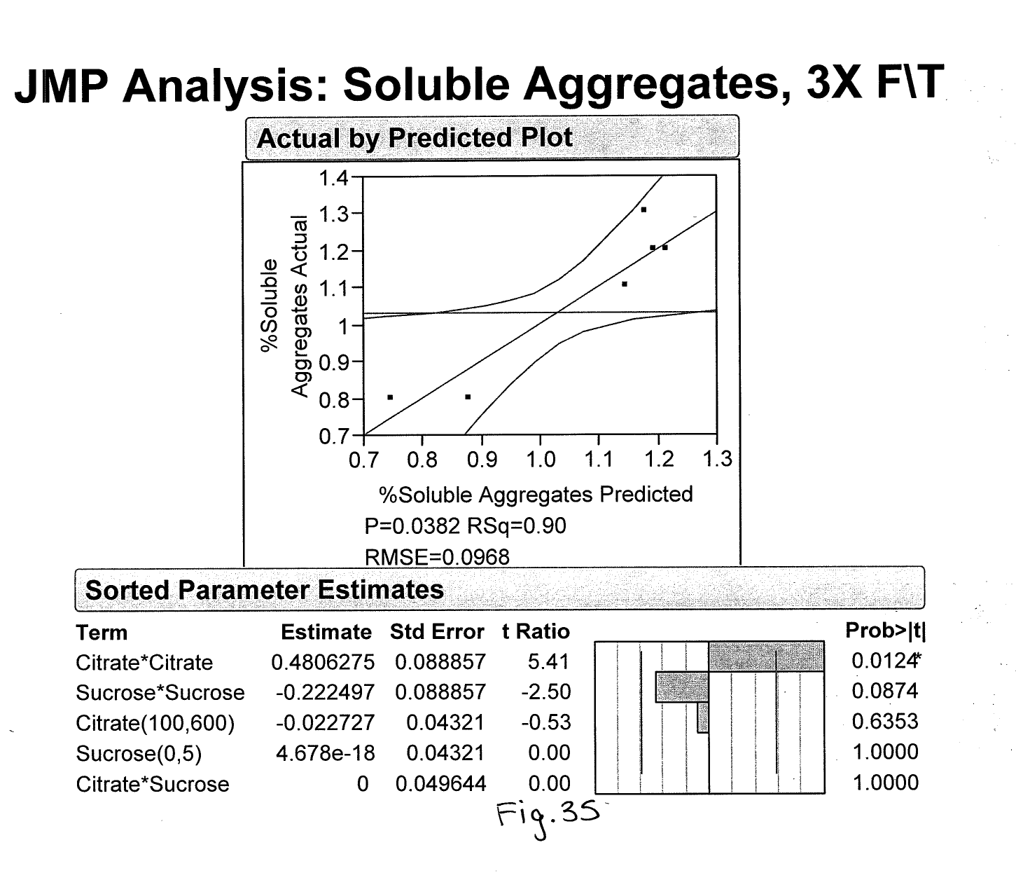

FIG. 36 provides results from an analysis of latent antithrombin.

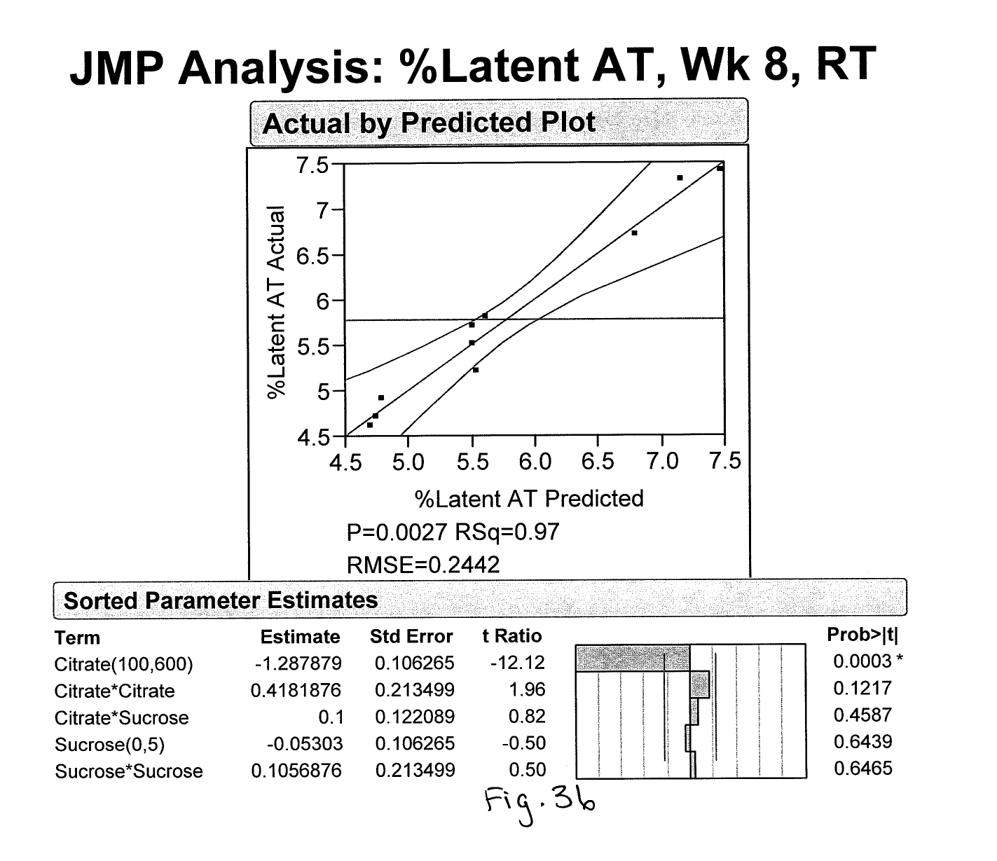

FIG. 37 provides results from an analysis of soluble aggregates.

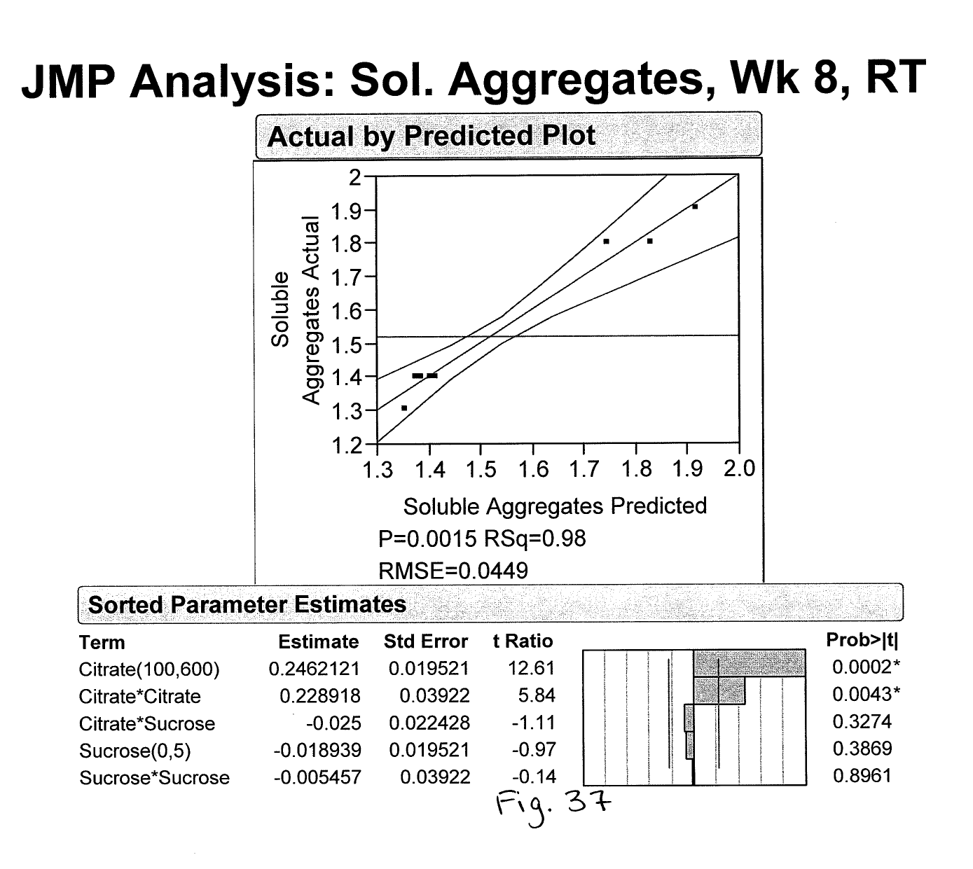

FIG. 38 shows a prediction profiler for two responses.

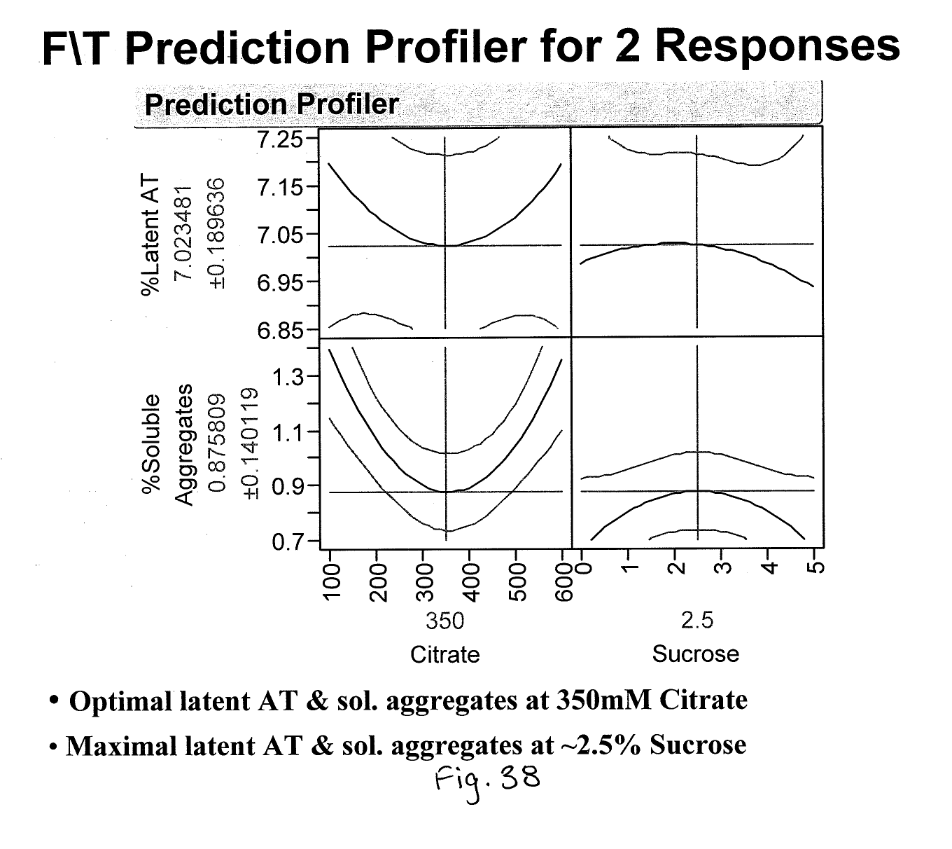

FIG. 39 shows a prediction profiler for two responses.

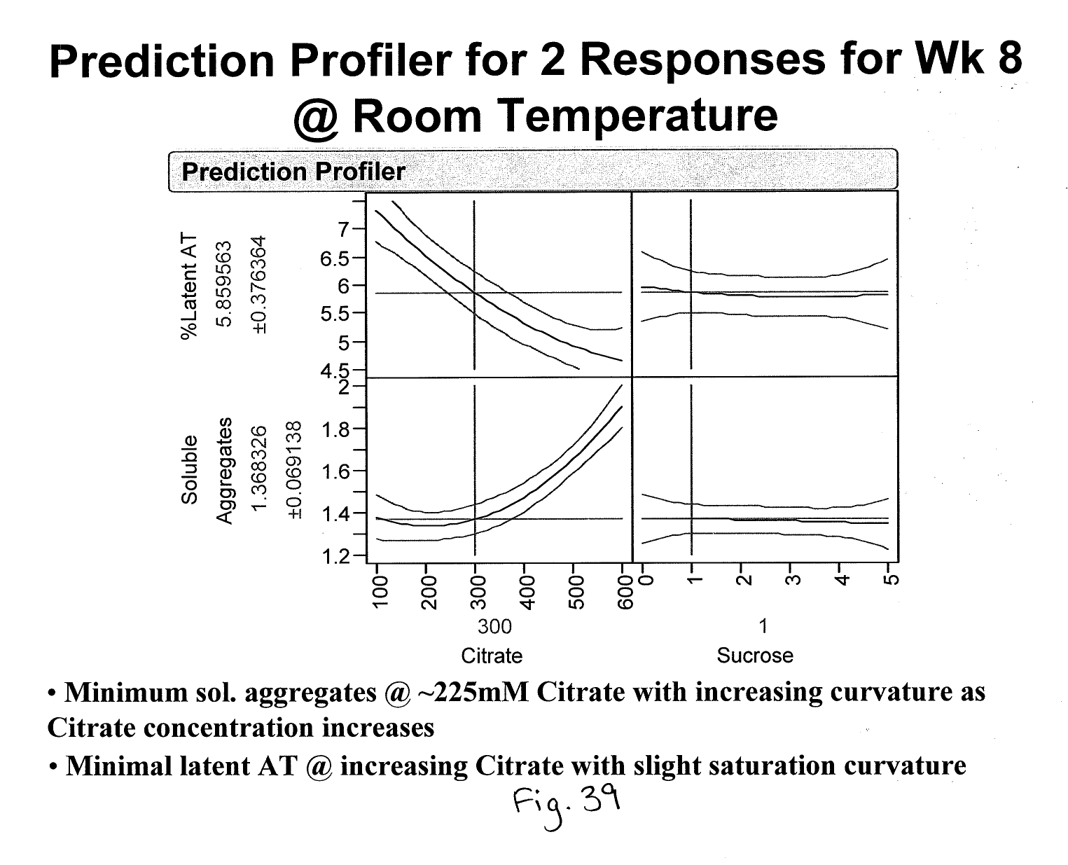

FIG. 40 provides another liquid stable formulation.

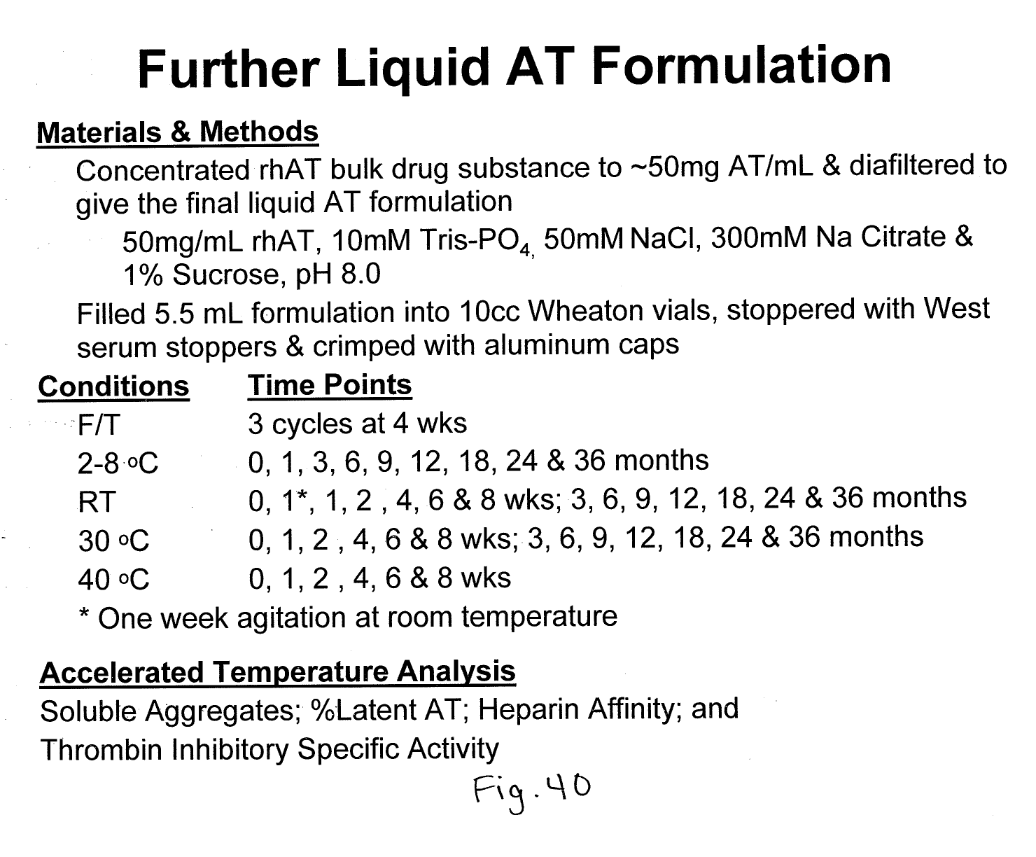

FIG. 41 provides a time plot for the liquid formulation.

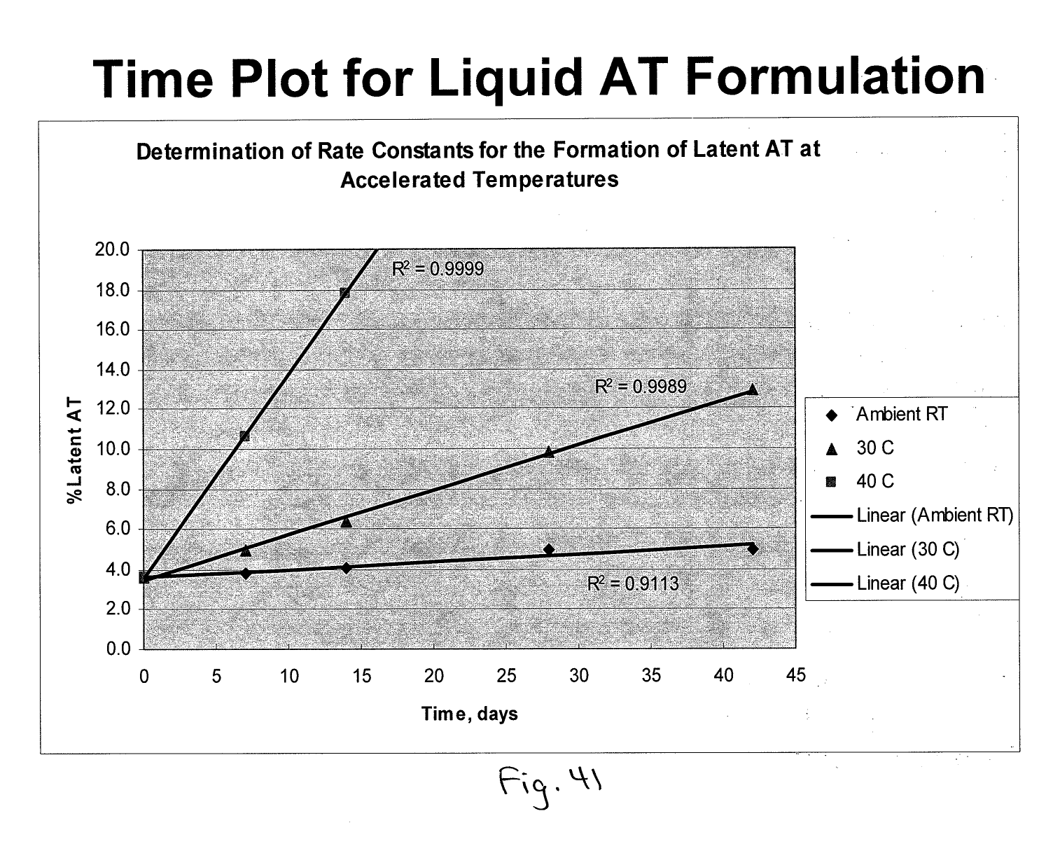

FIG. 42 provides another time plot for the liquid formulation.

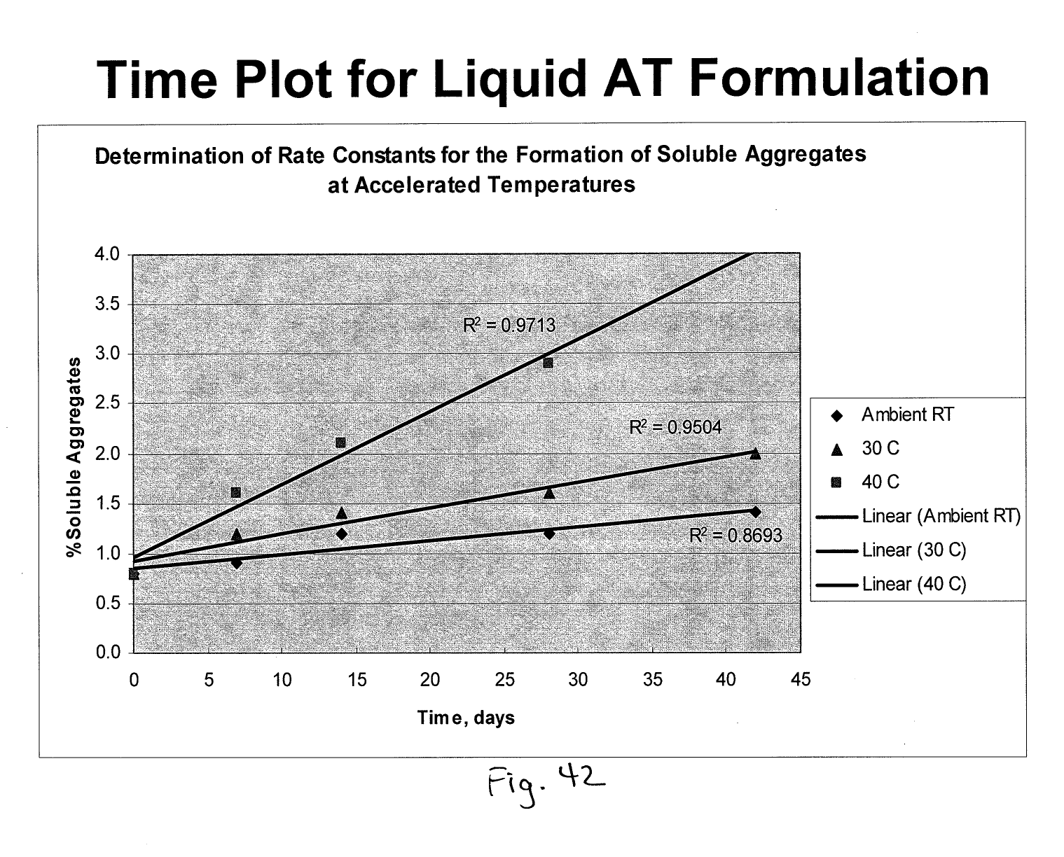

FIG. 43 provides an Arrhenius plot for the liquid formulation.

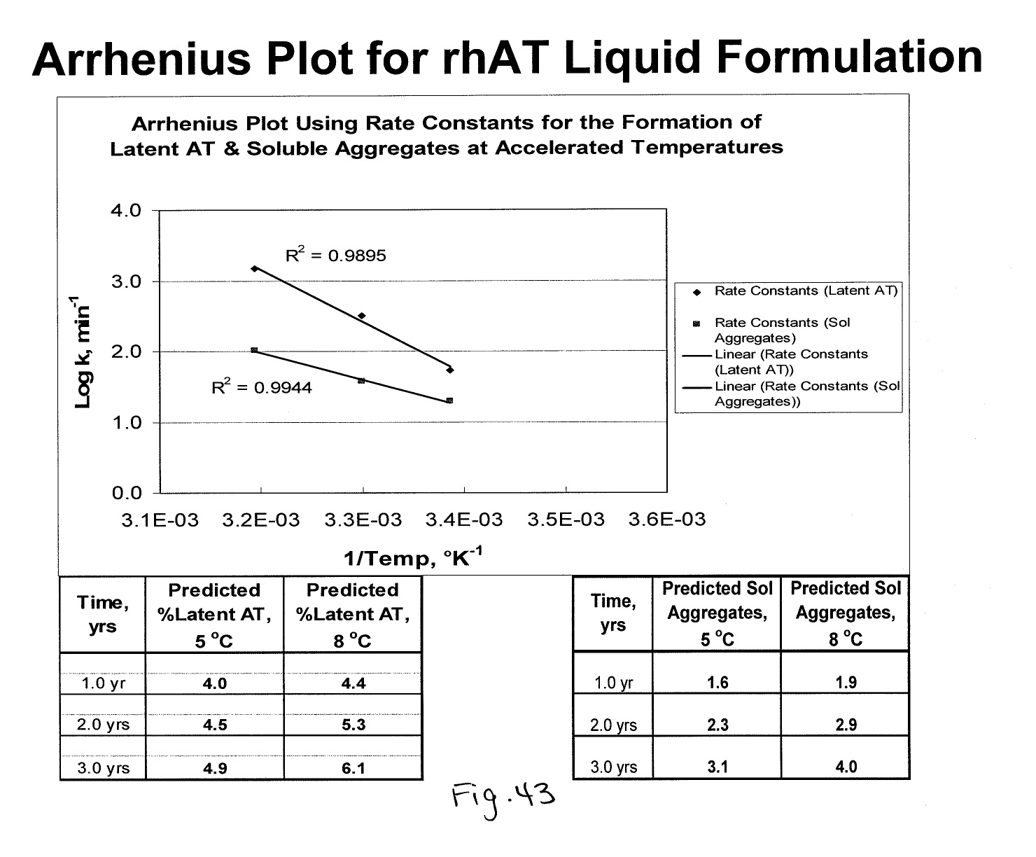

### DETAILED DESCRIPTION OF THE INVENTION

Therapeutic antithrombin (such as ATryn®) is stored as a lyophilized protein and reconstituted immediately prior to administration. There is a need for a stable liquid formulation of antithrombin, such as therapeutic antithrombin. In one aspect, the invention provides liquid formulations of antithrombin that allow for the prolonged storage of antithrombin without compromising the integrity or efficacy of the antithrombin.

### Antithrombin

Antithrombin is a glycoprotein of 432 amino acids and a molecular weight of 58 kDA. Antithrombin is a serine protease inhibitor that inhibits thrombin and Factor Xa. Antithrombin, as used herein refers to the alfa (or alpha) form of Antithrombin III. Antithrombin is naturally present in plasma and human antithrombin may be isolated from human plasma. Human antithrombin may also be produced by recombinant methods, resulting in recombinant human antithrombin (rhAT; unless specifically stated the term “antithrombin”, as used herein, includes rhAT).

Recombinant antithrombin alfa can be produced in transgenic animals and used to treat subjects deficient in antithrombin alfa (See e.g., U.S. Pat. No. 5,843,705, U.S. Pat. No. 6,441,145 and U.S. Pat. No. 7,019,193). ATryn® is a recombinantly produced human antithrombin alfa that is approved by the FDA for the prevention of peri-operative and peri-partum thromboembolic events in hereditary antithrombin deficient patients. In Europe, ATryn® is approved for use in surgical patients with congenital antithrombin deficiency for the prophylaxis of deep vein thrombosis and thromboembolism in clinical risk situations. ATryn® is provided as a lyophilized protein that needs to be reconstituted prior to administration.

In solution, antithrombin and rhAT have the potential to convert into a latent form that binds with low affinity to heparin and does not inhibit thrombin or Factor Xa. Without being bound by any theory, it is believed that the latent form of rhAT results from the exposure of the reactive loop in the protein to solvent, causing the less hydrophobic reactive center loop to re-orient to interact with the beta-pleated sheet in the protein. It was surprisingly found that the formulations presented herein minimize the formation of the latent (inactive) form of antithrombin.

The stable liquid formulations of antithrombin are suitable for administration intravenously, intraarterially or parenterally to patients in need of antithrombin therapy. Methods for administering the stable liquid formulations are, therefore, provided. The formulations are stable under readily available storage conditions. It was found that the new liquid formulations of antithrombin have a long shelf life and maintain the desired level of activity in these storage conditions during that time.

Furthermore, it was unexpectedly found that the presence of a stabilizing excipient, such as sodium citrate, in the formulation has a significant stabilizing effect on minimizing latent antithrombin and minimizing soluble aggregate formation, providing a formulation of antithrombin with a high-affinity binding to heparin.

It was also surprisingly found that in the antithrombin formulations described herein do not require a surfactant. In some circumstances, the addition of various surfactants causes increased oxidation of antithrombin. Not requiring the use of a surfactant may avoid the generation of harmful compounds, including hydrogen peroxide, which have a deleterious effect on the protein in some circumstances. (See e.g., Lavoie, J-C. et al. (1997) Pediatrics 99 (3): 1-5; Ding, S. (1993) J. Pharm. Biomed. Anal. 11(2):95-101). While many formulations comprising therapeutic proteins require surfactants, the formulations presented herein do not require the use of surfactants, although surfactants may be included in some embodiments.

It was also surprisingly found herein that the liquid formulations have a higher potency versus than currently available lyophilized formulations.

### Formulation

In one aspect the invention provides stable formulations of antithrombin (e.g., therapeutic antithrombin). A “stable” formulation of antithrombin as used herein has the following characteristics: soluble aggregates less than or equal to 5%, heparin affinity of greater than or equal to 80% and thrombin inhibitory specific activity of 5 to 9 IU/mg, after 1 year at 2 to 8 degrees Celcius. Ideally at 2-3 years at 2-8 degrees Celcius or room temperature.

The formulation of therapeutic antithrombin can include a range of concentration or weight of antithrombin. In some embodiments, the formulation includes 10 to 90 mg/ml of antithrombin, 20 to 80 mg/ml of antithrombin, 30 to 70 mg/ml of antithrombin, 40 to 60 mg/ml of antithrombin, 45 to 55 mg/ml of antithrombin, or 50 mg/ml antithrombin. In some embodiments, the formulation includes less than 10 mg/ml of antithrombin. In some embodiments, the formulation includes more than 90 mg/ml of antithrombin.

In some embodiments, the formulation includes a buffer. Buffers are added in order to allow for a stable pH. Suitable buffers for use in the present invention include phosphate buffers, bicarbonate buffers and tromethamine buffers. In one embodiment, the buffer is tromethamine (tris) buffer. In one embodiment the buffer is a tris-phosphate buffer. In some embodiments, the buffer is present in a concentration of between 1 mM and 100 mM, between 2 mM and 50 mM, or between 5 mM and 20 mM. In some embodiments, the buffer concentration is less than 1 mM. In some embodiments, the buffer concentration is more than 100 mM. In some embodiments, the buffer concentration is 10 mM. In other embodiments, the buffer concentration is 22 mM. It should be appreciated that the buffer concentration is dependent on the nature of the buffer that is being used. In some embodiments, the pH of the formulation is between pH 7 and pH 9 or between pH 7.5 and pH 8.5. In some embodiments, the pH of the formulation is 8.0. In some embodiments, the pH of the formulation is 7.5. In some embodiments, the pH of the formulation is 8.5. If needed, acid (such as HCL) or base (such as NaOH) can be added to the formulation to attain the desired pH.

The formulation of the present invention includes a stabilizing excipient, such as carboxylic acid or a salt thereof. In some embodiments, the carboxylic acid is sodium citrate. It was unexpectedly found that sodium citrate has a significant stabilizing effect on minimizing latent antithrombin alfa and soluble aggregate formation, resulting in high-affinity binding of the antithrombin alfa to heparin.

In some embodiments, the formulation includes a monocarboxylic acid and/or salt thereof. In some embodiments, the formulation includes a gluconic acid and/or sodium gluconate. In some embodiments, the formulation includes a dicarboxylic acid and/or a salt thereof. In some embodiments, the formulation includes a citric acid, succinic acid, malonic acid, maleic acid, tartaric acid and or a salt thereof. In some embodiments, the formulation includes a tricarboxylic aid and/or a salt thereof. In some embodiments, the formulation includes a nitrilotriacetic acid and/or sodium nitrilotriacetic acid. In some embodiments, the formulation includes a tetracarboxylic acid and/or salt thereof. In some embodiments, the formulation includes a ethylenediaminetetracetic acid (EDTA) and/or sodium EDTA. In some embodiments, the formulation includes a pentacarboxylic acid and/or a salt thereof. In some embodiments, the formulation includes a diethylenetriaminepentaacetic (DTPA) acid and/or sodium DTPA. Suitable carboxylic acids include, but are not limited to, citrate compounds, such as sodium citrate; tartrate compounds , succinate compounds, and EDTA. Kaushil et al in Protein Science 1999 8: 222-233 and Busby et al in the Journal of Biological Chemistry Volume 256, Number 23 pp 12140-1210-12147 describe carboxylic acids and their uses.

In some embodiments, the stabilizing excipient has a concentration of between 50 to 600 mM, between 250 to 500 mM, or between 250 to 350 mM. In some embodiments, the concentration of the stabilizing excipient is 300 mM. In some embodiments, the concentration of the stabilizing excipient is less than 100 mM. In some embodiments, the concentration of the stabilizing excipient is more than 600 mM. In one embodiment the stabilizing excipient is sodium citrate at a concentration of 300 mM.

In some embodiments, the formulation includes a sugar (e.g., a disaccharide sugar). The sugars have a stabilizing effect on the antithrombin and minimize aggregation of the therapeutic protein. Disaccharide sugars that can be added to the formulation, include but are not limited to sucrose, lactulose, lactose, maltose, trehalose and cellobiose. In some embodiments, the formulation includes sucrose or trehalose as the disaccharide.

In some embodiments, the sugar is present at between 0.5 to 5% (wt/volume). In some embodiments, the sugar is present at between 1 to 2%. In one embodiment, the sugar is present at 1%. In some embodiments, the sugar is present at less than 1%. In some embodiments, the sugar is present at more than 5%. In one embodiment, the sugar is sucrose or trehalose and is present at 1%.

In some embodiments, the formulation includes a salts. Salts that can be used in the formulations include sodium chloride and other physiological compatible salt. In some embodiments, the salt concentration is between 10 mM and 250 mM, between 25 mM and 100 mM. In some embodiments, the salt concentration is 50 mM. In some embodiments, the salt concentration is less than 10 mM. In some embodiments, the salt concentration is more than 250 mM. In some embodiments, the salt is sodium chloride and the concentration is 50 mM.

### Additives to Formulations

In some embodiments, the formulation includes one or more antioxidants. Antioxidants are substances capable of inhibiting oxidation by removing free radicals from solution. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfite, sodium bisulfite, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol and sodium sulfite. Such materials are typically added in ranges from 0.01 to 2.0%.

In some embodiments, the formulation includes one or more isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol.

In some embodiments, the formulation includes one or more preservatives. Suitable preservatives include but are not limited to: chlorobutanol (0.3-0.9% W/V), parabens (0.01-5.0%), thimerosal (0.004-0.2%), benzyl alcohol (0.5-5%), phenol (0.1-1.0%), and the like.

In some embodiments, the formulation includes one or more surfactants.

### Administration

The formulations of the present invention are primarily intended as a concentrated dosage for intravenous, intraarterial or parenteral administration. The formulations, therefore, are also primarily intended as a concentrated dosage for injection. In some embodiments, the concentration of the formulation is about 800 milliosmoles (mOsm) per kilogram. When administered to a patient, in one embodiment, the formulation of the present invention is diluted or can be diluted to about 290 to 320 mOsm per kilogram prior to administration.

The formulations described herein, when used in alone or in combination, are administered in therapeutically effective amounts. A therapeutically effective amount will be determined by the parameters discussed below; but, in any event, is that amount which establishes a level of the drug(s) effective for treating a subject, such as a human subject, having one of the conditions described herein (e.g., hereditary or acquired antithrombin deficiency). An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

The formulations described herein may include or be diluted into a pharmaceutically-acceptable carrier. The term “pharmaceutically-acceptable carrier” as used herein means one or more compatible solid, or semi-solid or liquid fillers, diluants or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carriers suitable for intravenous, intraarterial or parenteral, etc. formulations can be found in Remington's Pharmaceutical Sciences, Mack Publishing Company, Easton, Pa.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### Examples

A formulation (TR-0364-PRO) of antithrombin alfa (50 mg/ml) with the following components: 10 mM tris-phosphate, 300 mM sodium citrate, 50 mM sodium chloride, 1% (w/v) sucrose and pH 8 was evaluated for prolonged storage. After prolonged storage (over twelve months) at 2-8 degrees Celcius or room temperature, the characteristics of the formulations were evaluated. The evaluated characteristics include the ability to bind heparin and the ability to inhibit thrombin. The data presented show that formulations of the present invention have a significant stabilizing effect.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference with regard to the use or subject matter referenced herein.

We claim:

1. A formulation comprising: antithrombin, a buffer, a carboxylic acid, a disaccharide sugar and a salt.

2. The formulation of claim 1, wherein the formulation does not include a surfactant.

3. The formulation of claim 1, wherein the antithrombin is recombinant human antithrombin (rhAT).

4. The formulation of claim 1, wherein the antithrombin has a concentration of 45 to 55 mg/ml.

5. The formulation of claim 1, wherein the carboxylic acid is sodium citrate.

6. The formulation of claim 1, wherein the carboxylic acid has a concentration of 50 to 600 mM.

7. The formulation of claim 1, wherein the buffer is a tris-phosphate buffer.

8. The formulation of claim 1, wherein the buffer is between 1 mM and 100 mM.

9. The formulation of claim 1, wherein the pH is between 7.5 and 8.5.

10. The formulation of claim 1, wherein the disaccharide sugar is sucrose.

11. The formulation of claim 1, wherein the disaccharide sugar is 0.5 to 5% of volume by weight.

12. The formulation of claim 1, wherein the salt is sodium chloride.

13. The formulation of claim 1, wherein the salt has a concentration of between 25 mM and 100 mM.

14. The formulation of claim 1, wherein the antithrombin has a concentration of 50 mg/ml, wherein the carboxylic acid is sodium citrate and has a concentration of 300 mM, wherein the disaccharide sugar is sucrose and is 1% of volume by weight, wherein the salt is sodium chloride and has a concentration of 50 mM.

15. The formulation of claim 14, wherein the antithrombin is antithrombin alfa.

16. The formulation of claim 14, wherein the buffer is 10 mM tris-phosphate and the pH is 8.
